SEQUENCE LISTING (38 sequences)

SEQ ID NO: 1 - DNA, 24 bp, Homo sapiens
taaatcacgacgccgaccgctctt

SEQ ID NO: 2 - DNA, 23 bp, Homo sapiens
aaaacgcgaaccgcgcgtactca

SEQ ID NO: 3 - DNA, 23 bp, Homo sapiens
cctaaaaaccgctaacgacacta

SEQ ID NO: 4 - DNA, 25 bp, Homo sapiens
taaaccacgatataaaacgacactc

SEQ ID NO: 5 - DNA, 27 bp, Homo sapiens
tttgttgtttgtttattttttaggttt

SEQ ID NO: 6 - DNA, 26 bp, Homo sapiens
aacctaatactaccaattaaccccat

SEQ ID NO: 7 - DNA, 29 bp, Homo sapiens
gggatttgggaaagagggaaaggtttttt

SEQ ID NO: 8 - DNA, 26 bp, Homo sapiens
actaaaaactctaaaaaccccatccc

SEQ ID NO: 9 - DNA, 21 bp, Homo sapiens
yatcyggcygcgcyaacytay

SEQ ID NO: 10 - DNA, 15 bp, Homo sapiens
ctaacgcgccgaaac

SEQ ID NO: 11 - DNA, 110 bp, Homo sapiens
agggaatttttttttgtgatgttttggtgtgttagtttgttgtgtatattttgttgtggttttttttttggtttttttggttagttgtgtggtgattttggggattttag

SEQ ID NO: 12 - DNA, 110 bp, Homo sapiens
agggaattttttttcgcgatgtttcggcgcgttagttcgttgcgtatatttcgttgcggttttttttttggttttttcggttagttgcgcggcgatttcggggattttag

SEQ ID NO: 13 - DNA, 26 bp, Homo sapiens
aaactaacacaccaaaacatcacaaa

SEQ ID NO: 14 - DNA, 26 bp, Homo sapiens
gaactaacgcgccgaaacatcgcgaa

SEQ ID NO: 15 - DNA, 21 bp, Homo sapiens
yatcyggcygcgcyaacytay

SEQ ID NO: 16 - DNA, 16 bp, Homo sapiens
atcgccgcgcaactaa

SEQ ID NO: 17 - DNA, 26 bp, Homo sapiens
aatccccgaaatcgccgcgcaactaa

SEQ ID NO: 18 - DNA, 110 bp, Homo sapiens; identical to SEQ ID NO: 12

SEQ ID NO: 19 - DNA, 25 bp, Homo sapiens
gtttattaagacgttttatatttta

SEQ ID NO: 20 - DNA, 25 bp, Homo sapiens
gtttattaagatgttttatatttta

SEQ ID NO: 21 - DNA, 25 bp, Homo sapiens
tttttggatgttcgggtttttttag

SEQ ID NO: 22 - DNA, 25 bp, Homo sapiens
tttttggatgtttgggtttttttag

SEQ ID NO: 23 - DNA, 25 bp, Homo sapiens
ttgggtttttgcgtttaggaggttt

SEQ ID NO: 24 - DNA, 25 bp, Homo sapiens
ttgggtttttgtgtttaggaggttt

SEQ ID NO: 25 - DNA, 25 bp, Homo sapiens
gtaattagttacggaattttgaggt

SEQ ID NO: 26 - DNA, 25 bp, Homo sapiens
gtaattagttatggaattttgaggt

SEQ ID NO: 27 - DNA, 25 bp, Homo sapiens
gaaaaggttagcgttgtttttaaat

SEQ ID NO: 28 - DNA, 25 bp, Homo sapiens
gaaaaggttagtgttgtttttaaat

SEQ ID NO: 29 - DNA, 25 bp, Homo sapiens
ttagtttgttgcggattaaaatata

SEQ ID NO: 30 - DNA, 25 bp, Homo sapiens
ttagtttgttgtggattaaaatata

SEQ ID NO: 31 - DNA, 25 bp, Homo sapiens
tttatttttgtcgtgaatttaggga

SEQ ID NO: 32 - DNA, 25 bp, Homo sapiens
tttatttttgttgtgaatttaggga

SEQ ID NO: 33 - DNA, 25 bp, Homo sapiens
gaaggttaatttcgtttgtttgagt

SEQ ID NO: 34 - DNA, 25 bp, Homo sapiens
gaaggttaattttgtttgtttgagt

SEQ ID NO: 35 - DNA, 25 bp, Homo sapiens
aaaagatatttggcggaaatttgtg

SEQ ID NO: 36 - DNA, 25 bp, Homo sapiens
aaaagatatttggtggaaatttgtg

SEQ ID NO: 37 - DNA, 25 bp, Homo sapiens
ttattgagttgtgggagttggtatt

SEQ ID NO: 38 - DNA, 25 bp, Homo sapiens
ttattgagttgcgggagttggtatt

(19) United States
(12) Patent Application Publication
(10) Pub. No.: US 2007/0042365 A1
(43) Pub. Date: Feb. 22, 2007

(54) Assay for detecting methylation changes in nucleic acids using an intercalating nucleic acid

(76) Inventors: Douglas Spencer Millar, New South Wales (AU); John Robert Melki, New South Wales (AU); Geoffrey W. Grigg, New South Wales (AU); George L. Gabor Miklos, New South Wales (AU)

Correspondence Address: KNOBBE MARTENS OLSON & BEAR LLP, 2040 MAIN STREET, FOURTEENTH FLOOR, IRVINE, CA, 92614, US

(21) Appl. No.: 10/543,017

(22) PCT Filed: Jan. 23, 2004
(86) PCT No.: PCT/AU04/00083

§ 371 Date: Jul. 24, 2006

(30) Foreign Application Priority Data
Jan. 24, 2003 (AU) 2003900368

(51) Int. Cl. C12Q 1/68; C07H 21/04

(52) U.S. Cl. 435/6; 536/25.32

(57) ABSTRACT

A method for detecting the presence of a target nucleic acid in a sample including treating a sample containing nucleic acid with an agent that modifies unmethylated cytosine; providing to the treated sample a detector ligand in the form of an intercalating nucleic acid (INA) capable of binding to a target region of nucleic acid, and allowing sufficient time for the detector ligand to bind to the target nucleic acid, and detecting binding of the detector ligand to nucleic acid molecule in the sample to indicate the presence of the target nucleic acid.

### TECHNICAL FIELD

This invention relates to DNA hybridisation assays and to an improved oligonucleotide or intercalating nucleic acid (INA) assay. The invention relates particularly to methods for distinguishing specific base sequences including 5-methyl cytosine bases in DNA using these assays.

### BACKGROUND ART

A number of procedures were available for the detection of specific nucleic acid molecules. These procedures typically depend on sequence-dependent hybridisation between the target DNA and nucleic acid probes which may range in length from short oligonucleotides (20 bases or less) to sequences of many kilobases.

For direct detection, the target DNA is most commonly separated on the basis of size by gel electrophoresis and transferred to a solid support prior to hybridisation with a probe complementary to the target sequence (Southern and Northern blotting). The probe may be a natural nucleic acid or analogue such as INA or locked nucleic acid (LNA), PNA, HNA, ANA and MNA. The probe may be directly labelled (eg. with ³²P) or an indirect detection procedure may be used. Indirect procedures usually rely on incorporation into the probe of a “tag” such as biotin or digoxigenin and the probe is then detected by means such as enzyme-linked substrate conversion or chemiluminescence.

Another method for direct detection of nucleic acid that has been used widely is “sandwich” hybridisation. In this method, a capture probe is coupled to a solid support and the target DNA, in solution, is hybridised with the bound probe. Unbound target DNA is washed away and the bound DNA is detected using a second probe that hybridises to the target sequences. Detection may use direct or indirect methods as outlined above. The “branched DNA” signal detection system is an example that uses the sandwich hybridization principle (Urdea Ms Branched DNA signal amplification. Biotechnology 12: 926-928).

A rapidly growing area that uses nucleic acid hybridisation for direct detection of nucleic acid sequences is that of DNA micro-arrays (Young RA Biomedical discovery with DNA arrays. Cell 102: 9-15 (2000); Watson, New tools. A new breed of high tech detectives. Science 289:850-854 (2000)). In this process, individual nucleic acid species, that may range from oligonucleotides to longer sequences such as cDNA clones, were fixed to a solid support in a grid pattern. A tagged or labelled nucleic acid population was then hybridised with the array and the level of hybridisation with each spot in the array is quantified. Most commonly, radioactively or fluorescently-labelled nucleic acids (eg. cDNAs) were used for hybridisation, though other detection systems were employed.

The most widely used method for amplification of specific sequences from within a population of nucleic acid sequences is that of polymerase chain reaction (PCR) (Dieffenbach C and Dveksler G eds. PCR Primer A Laboratory Manual. Cold Spring Harbor Press, Plainview N.Y.). In this amplification method, oligonucleotides, generally 15 to 30 nucleotides in length on complementary DNA strands and at either end of the DNA region to be amplified, were used to prime DNA synthesis on denatured single-stranded DNA. Successive cycles of denaturation, primer hybridisation and DNA strand synthesis using thermostable DNA polymerases allows exponential amplification of the sequences between the primers. RNA sequences can be amplified by first copying using reverse transcriptase to produce a cDNA copy. Amplified DNA fragments can be detected by a variety of means including gel electrophoresis, hybridisation with labelled probes, use of tagged primers that allow subsequent identification (eg. by an enzyme linked assay), use of fluorescently-tagged primers that give rise to a signal upon hybridisation with the target DNA (eg. Beacon and TaqMan systems).

As well as PCR, a variety of other techniques have been developed for detection and amplification of specific sequences. One example is the ligase chain reaction (Barany F Genetic disease detection and DNA amplification using cloned thermostable ligase. Proc. Natl. Acad. Sci. USA 88:189-193 (1991)).

Currently the method of choice to detect methylation changes in DNA, such as were found in the GSTP1 gene promoter region in prostate cancer, were dependent on PCR amplification of such sequences after bisulfite modification of DNA. In bisulfite-treated DNA, cytosines were converted to uracils (and hence amplified as thymines during PCR) while methylated cytosines were non-reactive and remain as cytosines (Frommer M, McDonald L E, Millar D S, Collis C M, Watt F, Grigg G W, Molloy P L and Paul C L. A genomic sequencing protocol which yields a positive display of 5-methyl cytosine residues in individual DNA strands. PNAS 89: 1827-1831 (1992); Clark S J, Harrison J, Paul C L and Frommer M. High sensitivity mapping of methylated cytosines. Nucleic Acids Res. 22: 2990-2997 (1994)). Thus (after bisulfite treatment) DNA containing 5-methyl cytosine bases will be different in sequence from the corresponding unmethylated DNA. The Frommer et al 1992 results are the basis of the bisulfite method for sequencing 5-methyl cytosine residues in DNA. Several years later this assay was used as the basis of a PCR assay for the methylation status of CpG islands in U.S. Pat. No. 5,786,146. Primers may be chosen to amplify non-selectively a region of the genome of interest to determine its methylation status, or may be designed to selectively amplify sequences in which particular cytosines were methylated (Herman J G, Graff J R, Myohanen S, Nelkin B D and Baylin S B. Methylation-specific PCR: a novel PCR assay for methylation status of CpG islands. PNAS 93:9821-9826 (1996)).

Alternative methods for detection of cytosine methylation include digestion with restriction enzymes whose cutting is blocked by site-specific DNA methylation, followed by Southern blotting and hybridisation probing for the region of interest. This approach is limited to circumstances where a significant proportion (generally >10%) of the DNA is methylated at the site and where there is sufficient DNA, usually 10 μg, to allow for detection. Digestion with restriction enzymes whose cutting is blocked by site-specific DNA methylation, followed by PCR amplification using primers that flank the restriction enzyme site(s). This method can. utilise smaller amounts of DNA but any lack of complete enzyme digestion for reasons other than DNA methylation can lead to false positive signals.

Several years ago, peptide nucleic acids (PNA) in which the entire deoxyribose-phosphate backbone has been exchanged with a structurally homomorphous uncharged polyamide backbone composed of N-(2-aminoethyl)glycine units have been developed (Ray A and Norden B. Peptide nucleic acid (PNA): its medical and biotechnical applications and for the future. FASEB J 14: 1041-1060(2000)).

Methods have been developed utilizing PNA ligands for the sensitive and specific detection of DNA which do not require PCR amplification (WO 02/38801). Recently, a new DNA ligand, intercalating nucleic acid (INA), has been developed which has unique and useful properties.

The present inventors have developed new assays for detecting nucleic acids of interest using INA probes.

### DISCLOSURE OF INVENTION

In a first aspect, the present invention provides a method for detecting the presence of a target nucleic acid in a sample, the method comprising:
(a) treating a sample containing nucleic acid with an agent that modules unmethylated cytosine;
(b) providing to the treated sample a detector ligand in the form of an intercalating nucleic acid (INA) capable of binding to a target region of nucleic acid and allowing sufficient time for the detector ligand to bind to the target nucleic acid; and
(c) measuring binding of the detector ligand to a nucleic acid molecule in the sample to detect the presence of the target nucleic acid in a sample.

In a second aspect, the present invention provides a method for detecting methylation of a target nucleic acid in a sample, the method comprising:
(a) treating a sample containing nucleic acid with an agent that modifies unmethylated cytosine;
(b) providing to the treated sample a detector ligand in the form of an intercalating nucleic acid (INA) capable of distinguishing between methylated and unmethylated cytosine of nucleic acid and allowing sufficient time for a detector ligand to bind to a target nucleic acid; and
(c) detecting binding of the detector ligand to the nucleic acid in the sample such binding is indicative of the extent of methylation of the target nucleic acid.

In a third aspect, the invention provides a method for detecting the presence of a target nucleic acid in a sample, the method comprising:
(a) treating a sample containing nucleic acid with an agent that modifies unmethylated cytosine;
(b) providing a support to which is bound a capture ligand which is capable of recognising a first part of a target nucleic acid sequence;
(c) contacting the support with the treated sample for sufficient time to allow nucleic acid to bind to a capture ligand such that target nucleic acid in the sample binds to the support via the capture ligand;
(d) contacting the support with a detector ligand capable of recognising a second:.part of the target nucleic acid sequence and allowing sufficient time for a detector ligand to bind to a target nucleic acid bound to a support; and
(e) measuring binding of the detector ligand to nucleic acid bound to the support to determine the presence of the target nucleic acid in the sample, wherein at least one of the capture ligand or the detector ligand is in the form of an intercalating nucleic acid (INA).

In a fourth aspect, the present invention provides a method for estimating extent of methylation of a target nucleic acid in a sample, the method comprising:
(a) treating a sample containing nucleic acid wIth an agent that modifies unmethylated cytosine;
(b) providing a support to which is bound a capture ligand which is capable of recognising a first part of a target nucleic acid sequence;
(c) contacting the support with the treated sample for sufficient time to allow DNA to bind to a capture ligand such that target nucleic acid in the sample binds to the support via the capture ligand;
(d) contacting the support with a detector ligand capable of distinguishing between methylated and unmethylated cytosine of DNA such that the detector ligand binds to any target nucleic acid on the support; and
(e) detecting binding of the detector ligand to the support such that the degree or amount of binding is indicative of the extent of methylation of the target nucleic acid, wherein at least one of the capture ligand or the detector ligand is in the form of an intercalating nucleic acid (INA).

In a fifth aspect, the present invention provides a method for detecting a methylated CpG- or CpNpG-containing DNA, the method comprising:

(a) treating a sample containing DNA with bisulfite to modify unmethylated cytosine to uracil in the DNA;

(b) providing to the treated sample a detector INA ligand capable of distinguishing between methylated and unmethylated cytosine of DNA; and

(c) detecting the methylated DNA based on the presence or absence of binding of the detector INA ligand.

In a sixth aspect, the present invention provides a method for estimating extent of methylation of a target DNA in a sample, the method comprising:

(a) treating a sample containing DNA with bisulfite to modify unmethylated cytosine to uracil;

(b) providing a solid support in the form of a magnetic bead, multi-well mirotiter plate or shaped particle to which is bound a capture ligand in the form of an INA, PNA or oligonucleotide ligand which is capable of recognising a first part of a target DNA sequence;

(c) contacting the support with the treated sample suspected of containing the target DNA such that target DNA in the sample binds to the support via the capture ligand;

(d) contacting the support with a detector ligand in the form of an INA, PNA or oligonucleotide ligand capable of distinguishing between methylated and unmethylated cytosine of DNA; and

(e) determining the extent of methylation of the DNA bound to the support by measuring the amount of bound detector ligand, wherein at least one of the capture or detector ligands is an INA.

In one preferred form, the capture ligand is an INA. In another preferred form, the detector ligand is an INA.

In a seventh aspect, the present invention relates to use of an agent that modifies unmethylated cytosine but not methylated cytosine and one or more ligands in the form of an INA probes capable of distinguishing between methylated and unmethylated cytosine of nucleic acid in methods for assaying methylation of target nucleic acid.

In an eighth aspect, the present invention provides a kit for analysing nucleic acid which has been treated with an agent that modifies unmethylated cytosine comprising at least one INA ligand capable of distinguishing between methylated and unmethylated cytosine of DNA.

Preferably, the kit contains one or more INA ligands immobilized to a solid support. The kit may also contain primers for amplifying treated DNA.

The nucleic acid may comprise or be copied from the genomes of eukaryotes, prokaryotes and viruses, as well as mitochondrial nucleic acids, nucleic acids found in other cellular organelles and nucleic acids that are extracellular. Nucleic acids, as defined herein, may also include both DNA and RNA forms and natural or artificial derivatives thereof, such as intercalating nucleic acid (INA), Altritol Nucleic Acid (ANA), Cyclohexanyl Nucleic Acid (CNA), peptide nucleic acid (PNA), Locked Nucleic Acid (LNA), Hexitol Nucleic Acid (HNA), Manitol nucleic acid (MNA) and chimeric combinations thereof.

The nucleic acids may derive from normal or diseased organisms that have been infected by bacterial, viral, viroidor eukaryotic organisms or prions. The nucleic acids may also derive from modified (transgenic) organisms (irrespective of whether the modified organisms are made from germ line-, transient-, or somatic-transfection processes) that incorporate nucleic acids from different species or from artificially synthesized sources. The nucleic acids may derive from cells, tissues or organs of organisms with implanted or attached devices, these being mechanical, electronic. or chemical releasing (such as stents, patches, pacemakers etc). The nucleic acids may derive from organisms arising from non standard methods of conjugation, artificial insemination, cloning by embryonic stem cell methods, or by nuclear transfer, (from somatic or germ line nuclei), or by modification of cells or nuclei by cytoplasmic, nuclear or membranous extracts; or modification of cells by extraneous agents, (involving transdetermination and transdifferentiation processes), or from the input or modification of mitochondrial or other cellular organelles from the same or different species, or combinations thereof. The nucleic acid may derive from autologous-, allogeneic- or xeno-transplants; tissue or organ transplants; or tissues in which human cells have been transplanted into other organisms, (such as in model organism interventional cardiology). The nucleic acid may derive from organisms produced or modified by knock-out, knock-in or knock-down methods, (either in vivo, ex vivo, or by any method in which the genome or transcriptome is transiently or permanently altered, e.g., by RNAi, ribozyme, aptamer, transposon activation, drug or small molecule methodologies such as perturbations due to PNA, INA, ANA, MNA, LNA, HNA, CNA molecules or other nucleic acid-based conjugates, (including but not restricted to Trojan peptides). The nucleic acids may derive from all human life stages from fertilization to 48 hours post-mortem or from individuals at any stages of pregnancy, (normal or ectopic) and from embryonic or fetal material; as well as from individuals or organisms which are chromosomally imbalanced, or which are chimeras of different autologous cell populations, such as intersexes; or chimeras of diploid, aneuploid or segmentally aneuploid cell populations. The nucleic acids may derive from primary or cultured cell lines derived from any or all of the above sources, or from stored material such as histological specimens, tissues and organs as. well as from cells (and cell lines) and their derivatives isolated from human tissues, from autologous as well as allogeneic grafts, xenografts, as well as samples that may be derived from frozen or (otherwise stored; naturally or artificially preserved or mummified), dissected or resected sources; sources such as microscope slides, samples embedded in blocks or liquid media, or samples extracted from synthetic or natural surfaces or from liquids.

Preferably, the nucleic acid is DNA, more preferably genomic DNA from an animal or human.

The modifying agent is preferably selected from bisulfite, acetate or citrate. More preferably, the agent is sodium bisulfite, a reagent, which in the presence of water, modifies cytosine. into. uracil.

Sodium bisulfite (NaHSO₃) reacts readily with the 5,6-double bond of cytosine to form a sulfonated cytosine reaction intermediate which is susceptible to deamination, and in the presence of water gives rise to a uracil sulfite. If necessary, the sulfite group can be removed under mild alkaline conditions, resulting in the formation of uracil. Thus, potentially all cytosines will be converted to uracils. Any methylated cytosines, however, cannot be converted by the modifying reagent due to protection by methylation.

Intercalating nucleic acids (INA) are non-naturally occurring polynucleotides which can hybridize to nucleic acids (DNA and RNA) with sequence specificity. INA are candidates as alternatives/substitutes to nucleic acid probes in probe-based hybridization assays because they exhibit several desirable properties. INA are polymers which hybridize to nucleic acids to form hybrids which are more thermodynamically stable than a corresponding naturally occurring nucleic acid/nucleic acid complex. They are not substrates for the enzymes which are known to degrade peptides or nucleic acids. Therefore, INA should be more stable in biological samples, as well as, have a longer shelf-life than naturally occurring nucleic acid fragments. Unlike nucleic acid hybridization which is very dependent on ionic strength, the hybridization of an INA with a nucleic acid is fairly independent of ionic strength and is favoured at low ionic strength under conditions which strongly disfavour the hybridization of naturally occurring nucleic acid to nucleic acid. The binding strength of INA is dependent on the number of intercalating groups engineered into the molecule as well as the usual interactions from hydrogen bonding between bases stacked in a specific fashion in a double stranded structure. Sequence discrimination is more efficient for INA recognizing DNA than for DNA recognizing DNA.

Preferably, the INA is the phosphoramidite of (S)-1-O-(4,4′-dimethoxytriphenylmethyl)-3-O-(1-pyrenylmethyl)-glycerol.

INA are synthesized by adaptation of standard oligonucleotide synthesis procedures in a format.which is commercially available. Full definition of INA and their synthesis can be found in WO 03/051901, WO 03/052132, WO 03/052133 and WO 03/052134 (Unest A/S) incorporated herein by reference.

There are indeed many differences between INA probes and standard nucleic acid probes. These differences can be conveniently broken down into biological, structural, and physico-chemical differences. As discussed above and below, these biological, structural, and physico-chemical differences may lead to unpredictable results when attempting to use INA probes in applications were nucleic acids have typically been employed. This non-equivalency of differing compositions is often observed in the chemical arts.

With regard to biological differences, nucleic acids are biological materials that play a central role in the life of living species as agents of genetic transmission and expression. Their in vivo properties are fairly well understood. INA, however, is a recently developed totally artificial molecule, conceived in the minds of chemists and made using synthetic organic chemistry. It has no known biological function.

Structurally, INA also differs dramatically-from nucleic acids. Although both can employ common nucleobases (A, C, G, T, and U), the composition of these molecules is structurally diverse. The backbones of RNA, DNA and INA are composed of repeating phosphodiester ribose and 2-deoxyribose units. INAs differ from DNA or RNA in having one or more large flat molecules attached via a linker molecule(s) to the polymer. The flat molecules intercalate between bases in the complementary DNA stand opposite the INA in a double stranded structure.

The physico/chemical differences between INA and DNA or RNA are also substantial. INA binds to complementary DNA more rapidly than nucleic acid probes bind to the same target sequence. Unlike DNA or RNA fragments, INAs bind poorly to RNA unless the intercalating groups are located in terminal positions. Because of the strong interactions between the intercalating groups and bases on the complementary DNA strand, the stability of the INA/DNA complex is higher than that of an analogous DNA/DNA or RNA/DNA complex.

Unlike other DNA such as DNA or RNA fragments or PNAs, INAs do not exhibit self aggregation or binding properties.

In summary, as INA hybridize to nucleiacids with sequence specificity, INA are useful candidates for developing probe-based assays and are particularly adapted for kits and screening assays. INA probes, however, are not the equivalent of nucleic acid probes. Consequently, any method, kits or compositions which could improve the specificity, sensitivity and reliability of probe-based assays would be useful in the detection, analysis and quantitation of DNA containing samples. INAs have the necessary properties for this purpose.

In step (b), two detector ligands can be used where one ligand is capable of binding to a region of nucleic acid that contains one or more methylated cytosines and the other ligand capable of binding to a corresponding region of nucleic acid that before treatment (step (a)) contained no methylated cytosines. As a sample can contain many copies of a target nucleic acid, often the copies have different amounts of methylation. Accordingly, the ratio of binding of the two ligands will be proportional to the degree of niethylation of that nucleic acid target in the sample. The two ligands can be added together in the one test or can be added in separate duplicate tests. Each ligand can contain a unique marker which can be detected concurrently or separately in the one test or have the same marker and detected individually in separate tests.

Preferably, the capture ligand is selected from INA probe, PNA probe, LNA probe, HNA probe, ANA probe, MNA probe, CNA probe, oligonucleotide, modified oligonucleotide, single stranded DNA, RNA, aptamer, antibody, protein, peptide, a combination thereof, or chimeric versions thereof.

More preferably, the capture ligand is an INA probe, PNA probe or an oligonucleotide probe. Even more preferably, the capture ligand is an INA probe.

The support can be any suitable support such as a plastic materials, fluorescent beads, magnetic beads, shaped particles, plates, microtiter plates, synthetic or natural membranes, latex beads, polystyrene, column supports, glass beads or slides, nanotubes, fibres or other organic or inorganic supports. Preferably, the support is a magnetic bead, a fluorescent bead, a shaped particle or a microliter plate with one or more wells.

The solid substrate is typically glass or a polymer, the most commonly used polymers being cellulose, polyacrylamide, nylon, polystyrene, polyvinyl chloride or polypropylene. The solid supports may be in the form of tubes, beads, discs or microplates, or any other surface suitable for conducting an assay. The binding processes are well-known in the art and generally consist of cross-linking covalently binding or physically adsorbing the molecule to the insoluble carrier.

In a preferred form, step (b) comprises a plurality of capture ligands arrayed on a solid support. The array may contain multiple copies of the same ligand so as to capture the same target nucleic acid on the array or may contain a plurality of different ligands targeted to different nucleic acid so as to capture a plurality of target nucleic acid molecules on the array. Typically, the array contains from about 10 to 200,000 capture ligands. It will be appreciated, however, that the array can have any number of capture ligands.

In one form, capture oligonucleotide probes, INA probes, or capture PNA probes can be placed on an array and used to capture bisulfite-treated nucleic acid to measure ethylated states of nucleic acid. Array technology is well known and has been used to detect the presence of genes or nucleotide sequences in untreated samples. The present invention, however, can extend the usefulness of array technology to provide valuable information on methylation states of many different sources of nucleic acid.

In a preferred form the sample can be any biological sample such as stem cells, blood, urine. faeces, semen, cerebrospinal fluid, cells or tissue such as brain, colon, urogenital, lung, renal, hematopoietic, breast, thymus, testis, ovary, or uterus, environmental samples, microorganisms including bacteria, virus, fungi, protozoan, viroid and the like. Stems cells include populations of cells containing true progenitor cells. This also applies to germ cell populations and also includes stem cells that fuse with somatic cells to form hybrid cells capable of adopting a particular phenotype.

Preferably, the modifying agent is capable of modifying unmethylated cytosine but not methylated cytosine. The agent is preferably is selected from bisulfite, acetate and citrate. Preferably, the agent is sodium bisulfite and cytosine is modified to uracil.

The term “modifies” as used herein means the conversion of an unmethylated cytosine to another nucleotide which will distinguish the unmethylated from the methylated cytosine. Preferably, the agent modifies unmethylated cytosine to uracil. Preferably, the agent used for modifying unmethylated cytosine is sodium bisulfite. Other agents that similarly modify unmethylated cytosine, but not methylated cytosine can also be used in the method of the invention. Examples include, but not limited to bisulfite, acetate or citrate. Preferably, the agent is sodium bisulfite, a reagent, which in the presence of water, modifies cytosine into uracil.

Sodium bisulfite (NaHSO₃) reacts readily with the 5,6-double bond of cytosine, but poorly with methylated cytosine. Cytosine reacts with the bisulfite ion to form a sulfonated cytosine reaction intermediate which is susceptible to deamination, giving rise to a sulfonated uracil. The sulfonate group can be removed under alkaline conditions, resulting in the formation of uracil. Thus all unmethylated cytosines will be converted to uracil while methylated cytosines will be protected from conversion so that ligands can be prepared that will recognise sequences containing cytosine or corresponding sequences. containing uracil. The ratio of binding of the two probes can provide an accurate measure of the degree of methylation in a given nucleic acid.

Importantly, in many situations there is no need to amplify the nucleic acid to obtain the required information thus overcoming potential errors and resulting in a faster and more simple assay amenable to automation. Amplification after capture or nucleic acid selection prior to treatment is also possible for the present invention.

In a preferred form, the detector ligand is directed to a CpG- or CpNpG-containing region of DNA, where N designates any one of the four possible bases A, T, C, or G. Preferably, the CpG- or CpNpG-containing region of DNA is in a regulatory region of a gene or an enhancer of any regulatory element or region including promoter, enhancer, oncogene, retro-element, mobile or mobilisable sequence or other regulatory element which activity is altered by environmental factors including chemicals, toxins, drugs, radiation, synthetic or natural compounds and microorganisms or other infectious agents such as viruses, bacteria, fungi and prions. For example, the promoter or regulatory element can be a tumour suppressor gene promoter, oncogene or any other element or region that may control or influence one or more genes implicated in a disease state or changing normal state such as aging.

The presence of methylated CpG- or CpNpG-containing region of DNA in a specimen can be indicative of a cell functional change, particularly as regards cell reprogramming. The change may also be a proliferative disorder. It can include low grade astrocytoma, anaplastic astrocytoma, glioblastoma, medulloblastoma, colon cancer, lung cancer, renal cancer, leukemia, breast cancer, prostate cancer, endometrial cancer and neuroblastoma, or disturbances in normal cell division, differentiation or metabolism/catabolism of stem cell populations.

In order to assist in the reaction of the nucleic acid modifying agent, optional additives such as urea, methoxyamine and mixtures thereof can be added.

Step (b) is typically used to capture a nucleic acid of interest which will be analysed for methylation in subsequent steps of the method. Thus, step (b) allows the capture and concentration of nucleic acid of interest. Preferably one or more INA probes are used in step (b).

In one preferred form, step (b) comprises a plurality of capture ligands arrayed on a solid support. The array may contain multiple copies of the same ligand so as to capture the same target nucleic acid on the array for subsequent testing. Alternatively, the array may contain a plurality of different capture ligands targeted to different DNA molecules so as to capture many different target DNA samples on the array for subsequent testing. In a preferred form, the capture ligands are bound to wells of a micrtiter plate so that multiple assays may be carried out.

In step (d), two detector ligands can be used where one ligand is capable of binding to a region of nucleic acid that contains one or more methylated cytosines and the second ligand is capable of binding to a corresponding region of nucleic acid that contains no methylated cytosines. A sample can contain many copies of a target nucleic acid with the copies having different amounts of methylation. Accordingly, the ratio of binding of the two ligands will be proportional to the degree of methylation of that nucleic acid target in the sample. The two ligands can be added together in the one test or can be added in separate duplicate tests. Each ligand can have an unique marker which can be detected concurrently or separately in the one test or have the same marker and detected individually in separate tests.

In order to detect binding of the detector ligand to a target nucleic acid, preferably the ligand has a detectable label attached thereto. The presence of bound label being indicative of the extent of binding of the ligand. Suitable labels include, but not limited to, chemiluminescence, fluorescence, radioactivity, enzyme, hapten, and dendrimer.

The detector ligands used in the present invention for detecting CpG- or CpNpG-containing DNA in a sample, after bisulfite modification, can specifically distinguish between untreated DNA, methylated, and unmethylated DNA. Detector ligands in the form of oligonucleotide or PNA or INA probes for the non-methylated DNA preferably have a T or A in the 3′ CpG or CpNpG pair to distinguish it from the C retained in methylated DNA.

The probes of the invention can be designed to be “substantially” complementary to one strand of the genomic locus to be tested and include the appropriate G or C nucleotides. This means that the primers should be sufficiently complementary to hybridize with a respective region of interest under conditions which allow binding. In other words, the probes preferably should have sufficient complementarity with the 5′ and 3′ flanking sequences to hybridize therewith.

The INA probes of the invention may be prepared using any suitable method known to the art. Preferably, the probes are prepared in accordance with the teaching of WO 03/051901, WO 03/052132, WO 03/052133 and WO 03/052134 (Unest A/S) incorporated herein by reference.

The methods according to the present invention relating to methylation states of target nucleic acid can use any nucleic acid sample, in purified or unpurified form, as the starting material, provided it contains, or is suspected of containing, the specific nucleic acid sequence containing the target region (usually CpG br CpNpG). In one preferred form, unamplified samples are used in the methods according to the present invention.

INA mixtures or specific INA molecules can be used in an amplification enrichment step prior to capture by the detector ligand. Single or large numbers of INAs could be used for specific or random amplification of bisulphite-treated nucleic acid.

Nucleic acid molecules of interest may be selected or concentrated prior to step (a) of the methods according to the present invention. An enrichment or selection step icludes, bit not limited to, physical methods including sonication and shearing, enzymatic digestion, enzymatic treatment, restriction digestion, nuclease treatment, Dnase treatment, concentration, antibody capture, chemical methods including acidic or base digestion and combinations thereof. For example, an antibody directed 5-methyl cytosine may be used to capture nucleic acid such as genomic DNA rich in 5-methyl cytosines or highly methylated. The nucleic acid can be derived from genomic DNA which has undergone cleavage by any suitable physical or enzymatic means in order to break it up into more manageably sized nucleic acid.

The nucleic acid-containing specimen used for detection of methylated CpG or CpNpG may be from any source and may be extracted by a variety of techniques such as that described by Maniatis, et al (Molecular Cloning: A Laboratory Manual, Cold Spring Harbor, N.Y., pp 280, 281, 1982).

Where the nucleic acid in the sample contains two strands, it is necessary to separate the strands of the nucleic acid before it can be modified. Strand separation can be effected either as a separate step or simultaneously with chemical treatment. This strand separation can be accomplished using various suitable denaturing conditions, including physical, chemical, or enzymatic means, the word “denaturing” includes all such means. One physical method of separating nucleic acid strands involves heating the nucleic acid until it is denatured. Typical heat denaturation may involve temperatures ranging from about 80° to 105° C. for times ranging from about 1 to 10 minutes for DNA. Strand separation may also be induced by an enzyme from the class of enzymes known as helicases or by the enzyme RecA, which has helicase activity, and in the presence of riboATP, is known to denature DNA. The reaction conditions suitable for strand separation of DNA with helicases were described by Kuhn Hoffmann-Berling (CSH-Quantitative Biology, 43:63, 1978) and techniques for using RecA were reviewed in C. Radding (Ann. Rev. Genetics, 16:405-437, 1982).

The detectable label may be chemiluminescent, fluorescent, or radioactive or contain a second label or marker in the form of a microsphere, or nanocrystal. The fluorescent or radioactive microsphere or nanocrystal may be covalently bound to the capture or detector ligand.

Preferably the specificity of hybridization to target nucleic acid is used to discriminate between methylated cytosines and unmethylated cytosines.

Many suitable fluorochromes that bind to nucleic acid, some selective for single-stranded DNA, and that differ in their excitation and emission wavelengths were known. The detection system could also be an enzyme carrying a positively charged region that will selectively bind to the DNA and that can be detected using an enzymatic assay, or a positively charged radioactive molecule that binds selectively to the captured DNA. The suitable entity may also be. coreishell CdSe/ZnS semiconductor nanocrystals (Gerion et al 2002 J Am Chem Soc 24:7070-7074).

Using INA probes as one of the ligands in this procedure has very significant advantages over the use of oligonucleotide or PNA probes. INA binding reaches equilibrium faster and exhibits greater sequence specificity and, as INAs carry one or more intercalating groups, they bind the target DNA molecules with a higher binding coefficient than other ligands such as oligonucleotides or PNAs. The binding characteristics can be modified by choosing different numbers of intercalating groups to add to the INA.

As the present invention can use direct detection methods, the methods can give a true and accurate measure of the amount of a target nucleic acid in a sample. The methods are not confounded by potential bias inherent in prior art methods that rely for signal amplification on processes such as PCR, where the enzymes commonly used in such procedures can introduce systematic bias through differential rates of amplification of different sequences.

There are a number of detector systems and instruments available for detecting or measuring fluorescence or radioactivity. Improvements and advancement in instrumentation are being made by a number of manufacturers. It will be appreciated that many different measuring instruments can be used for the present invention. For example, Multi Photon Detection is a proprietary system for the detection of ultra low amounts of selected radioisotopes. It is 1000 fold more sensitive than existing methods. It has a sensitivity of 1000 atoms of iodine 125, with quantitation of zeptomole amounts of biomaterials. It requires less than 1 picoCurie of isotope which is 100 times less activity than in a glass of water. A family of MPD instruments already exists for measuring radioactivity in a sample. They consist of instruments that are configured for 96 well, 384 well and higher. MPD uses coincident multichannel detection of photons coupled with computer controlled electronics to selectively count only those photons that are compatible with an operator-selected radioisotope. As many different isotopes can be used, this is a multicolor system. The MPD imager system is at least 100 fold more sensitive than a phosphor imager. Such instrumentation would be particularly suitable in the detection part of the present invention where ligands or supports are made radioactive.

Beads containing capture or detector ligands bound thereto can be processed or measured by cell sorters which measure fluorescence. Examples or suitable instruments. include flow cytometers and modified versions thereof.

The methods according to the present invention are particularly suitable for scaling up and automation for processing many samples.

Notwithstanding the above, the methods described can be used in conjunction with such amplification procedures if it is necessary to amplify limiting amounts of DNA in order provide enough material for detection. In addition, PCR may be used to selectively amplify DNA that has been captured with an INA ligand directed to methylated or unmethylated nucleic acid.

Methylated DNA: In a particular adaptation as detailed in the present invention, the methods can. be used to distinguish the presence of methylated cytosines in DNA that has been treated with sodium bisulfite. As cytosines were converted to uracils while methyl cytosines remain unreacted, the sequence of bisulfite-treated DNA derived from methylated and unmethylated molecules is different. By choosing a specific INA ligand (4 to 100 residues long, preferably 20±10 residues long) to selected target regions the specificity of hybridisation can be used to discriminate between methylated cytosines at CpG or CpNpG sites (which remain as cytosines) and unmethylated CpG or CpNpG sites where the cytosine is converted to uracil, while ensuring that only molecules in which cytosines that were not in CpG or CpNpG sites have fully reacted and been converted to uracils were assessed.

Methylated cytosines at other sites can similarly be detected. Appropriate INA probes can be used as controls to identify the presence of molecules that have not reacted completely with bisulfite (one or more cytosines not converted to uracil). It will be appreciated, however, that other ligands which can differentiate between the methylation states of DNA can be used in a similar manner.

The methods were amenable for use in a variety of formats including multiwell plates, micro-arrays, fiber optic arrays and particles in suspension. The appropriate selection of specific ligands for use in an array format can allow for the simultaneous determination of the methylation state of individual cytosines in multiple target regions.

Polymorphism/mutation and epimutation detection: The methods according to the present invention can be applied to the discrimination of different alleles of a gene where the sequence of the capture ligand and/or the detector ligand will match with one allele but mismatch with the other.

DNA Quantification: By using the methods according to the present invention, it is possible to directly determine within a DNA population the proportion of molecules having one sequence versus another at a particular region. This can be done by coupling ligands representing the alternate forms of the sequence to supports such as microspheres charged with differently coloured fluorochromes, nanocrystals or radioactive molecules, particles and microtiter plates. Such differences in sequence may be differences in the original base sequence of the gene or differences in base sequence in bisulfite-treated DNA that were due to differences in methylation in the original DNA.

Cell quantification: The methods can be applied to determining the ratio of cells in a population (such as in cancer and normal cells) that differ in base sequence at a particular site in the genome.

Variations: The methods were amenable for use in a variety of formats including multiwell plates, micro-arrays, fiber optic arrays and particles in suspension. The appropriate selection of specific INA probes for use in an array format can allow for the simultaneous determination of the presence of different DNA sequences, eg. for the determination of the methylation state of individual cytosines in multiple target regions.

Throughout this specification, unless the context requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia before the priority date of each claim of this application.

In order that the present invention may be more clearly understood, preferred forms will be described with reference to the following drawings and examples.

### BRIEF DESCRIPTION OF THE DRAWINGS

FIG. 1 shows sandwich INA signal amplification

FIG. 2 shows sandwich INA signal amplification technology using magnetic beads or detectable particles.

FIG. 3 shows a schematic of the capture of methylated DNA using antibody.

FIG. 4 shows a schematic of the detection of methylated DNA using microspheres

FIG. 5 shows a schematic of the detection of methylated DNA using microspheres.

FIG. 6 shows results of enrichment factor provided when comparing genomic DNA samples that did not receive antibody versus antibody capture samples.

FIG. 7 shows results of non-PCR signal amplification using the antibody capture multiple ligand assay. The results show signals obtained using 1. no antibody enrichment with LNCaP DNA (methylated DNA), 2. Antibody enriched Du145 DNA (unmethylated DNA) and 3. Antibody enriched LNCaP DNA (methylated DNA).

FIG. 8 shows a schematic of shows a schematic of the detection of methylated DNA and subsequent amplification.

FIG. 9 shows agarose gel representation of the INA capture and PCR method. INA ligands specific for an unmethylated genomic DNA sequence were coupled to magnetic beads and were mixed with genomic bisulphite treated DNA. The bead/DNA complex was washed and the bound molecules used as a template in PCR for a downstream region.. LANES; MARKER, 1, 2, 3.

LANE 1: HepG2 DNA (Known to be methylated at target site),

LANE 2: Du145 DNA (Known to be unmethylated at target site),

LANE 3: BL13 DNA (Known to be unmethylated at target site)

FIG. 10 shows specificity of an INA directed against unmethylated DNA. The graph shows the percentage methylation of the DNA.

FIG. 11 shows specificity of an INA directed against methylated DNA. The graph shows the percentage methylation of the DNA.

FIG. 12 shows the signals generated on hybridization of the PNA, INA and oligo samples with a synthetic 110 bp oligo designed to a methylated region of the GSTPI gene. The oligo was diluted as described then labelled and hybridised to the samples. As can be seen, the INA gave signal intensities similar if not higher than the PNA ligand.

FIG. 13 shows Hybridisation results using INAs versus conventional oligonucleotides. Top two rows signals generated using INAs. Bottom two rows signals grated using conventional oligonucleotides. From the results, the superior quality of the hybridisation signals generated using INA ligands can be clearly seen.

### MODE(S) FOR CARRYING OUT THE INVENTION

### DEFINITIONS

Epigenetics/Epigenomics/Methylomics

The analysis of 5-methyl cytosine residues in nucleic acids from samples of human, animal, bacterial (including nanobacterial and extracellular as well as intracellular bacteri a) and viral origins at all life cycle stages, in all cells, tissues and organs from fertilization until 48 hours post mortem, and cells (and cell lines) and their derivatives isolated from human tissues, autologous as well as non-autologous grafts, xenografts, as well as samples that may be derived from frozen or (otherwise stored) dissected or resected sources, histological sources such as microscope slides, samples embedded in blocks or liquid media, or samples extracted from synthetic or natural surfaces or from liquids.

Epigenetics/Epigenomics/Methylomics

Includes 5-methyl cytosine analyses of the naturally occurring variation between cells, tissues and organs of healthy individuals, (health as defined by the WHO), as well as cells, tissues and organs from diseased individuals, (diseased in this sense including all human diseases, afflictions, ailments and deviant conditions described or referred to in Harrison's Principles of Internal Medicine, 12th Edition, edited by Jean D Wilson et al., McGraw Hill Inc, and subsequent later editions; as well as all diseases, afflictions ailments and deviant conditions described in OMIM, Online Mendelian Inheritance in Man, www.ncbi.gov and therein), but with emphases on the leading causes of death, namely, malignant neoplasms, (cancer), ischaemic heart disease, cerebrovascular disease, chronic obstructive pulmonary disease, pneumonia and influenza, diseases of arteries, (including atherosclerosis and aortic aneurysm), diabetes mellitus, and central nervous system diseases, together with socially debilitating conditions such as anxiety, stress related neuropsychiatric conditions and obesity, and all conditions arising from abnormal chromosome number or chromosome rearrangemerits, (aneuploidy involving autosomes as well as sex chromosomes, duplications, deficiencies, translocations and insertions in germ line or somatic conditions), as well as similar abnormalities of the mitochondrial genomes.

The normal or diseased individuals may be from, (a), populations of diverse ethnicity and evolutionary lineages (b), strains and geographical isolates (c), sub species, (d), twins or higher order multiplets of the same or different sex, (e), individuals arising from normal methods of conjugation, artificial insemination, cloning by embryonic stem cell methods, or by nuclear transfer, (from somatic or germ line nuclei), or by modification of cells or nuclei by cytoplasmic extracts or by extraneous agents, (transdetermination and transdifferentiation), or from the input or modification of mitochondrial or other cellular organelles, (f), individuals deriving from transgenic knock-out, knock-in or knock-down methods, (either in vivo, ex vivo, or by any method in which gene activity is transiently or permanently altered, e.g., by RNAi, ribozyme, transposon activation, drug or small molecule methodologies, PNA, INA, AMA, AHA, etc . . . or nucleic acid based conjugates, (including but not restricted to Trojan peptides), or individuals at any stages of pregnancy, (normal or ectopic).

Epigenetics/EpigenomicslMethylomics

Means analyses of 5-methyl cytosine residues in nucleic acid from prokaryotic or eukaryotic organisms and viruses (or combinations thereof), that are associated with human diseases in extracellular or intracellular modes, for the purposes of determining, and therapeutically altering, in both normally varying and diseased systems, the changed parameters and underlying mechanisms of:

(i) gerietic diseases;

(ii) non-genetic or epigenetic diseases caused by environmentally induced factors, be they of biological or non-biological origin, (environmental in this sense being taken to also include the environment within the organism itself, during all stages of pregnancy, or under conditions of fertility and infertility treatments);

(iii) predisposition to genetic or non genetic diseases, including effects brought about by the “prion” class of factors, by exposure to pressure changes and weightlessness, or by radiation effects;

(iv) 5-methyl cytosine changes in the processes of aging in all cell types, tissues, organ systems and biological networks, including age related depression, pain, neuropsychiatric and neurodegenerative conditions and pre- and post-menopausal conditions, (including reduced fertility; in both sexes);

(v) 5-methyl cytosine changes in cancer (or diseases underpinned by somatic changes in nucleic acid dosages; including changes in cells with abnormal karyotypes arising from nucleic acid amplification, deletion, rearrangement, translocation and insertion events), and their variations or alterations in different cell cycle phenomena (including cell cycle effects on diurnal rhythms, photoperiod, sleep, memory, and “jet lag”;

(vi) 5-methyl cytosine changes in metabolic networks defined in the broadest sense, from the zygote through embryogenesis, foetal development, birth, adolescence, adulthood and old age (including metabolic effects brought about by hypoxia, anoxia, radiation of any type, (be it ionizing or non ionizing, or arising from chemotherapeutic treatments, or high altitude exposure), stress, or by imbalances between the mitochondrial, nuclear or organellar genomes;

(vii) 5-methyl cytosine alterations due to responses at the molecular, cellular, tissue, organ and whole organism levels to proteins, polypeptides, peptides, and DNA, RNA, PNA, INA, AMA, AHA, etc. or peptide aptamers (including any with post translational additions, post translational cleavage products, post translational modifications (such as inteins and exeins, ubiquination and degradation products); proteins, polypeptides and peptides containing rare natural amino acids, as well as single rare amino acids such as D-serine involved in learning, brain growth and cell death; drugs, biopharmaceuticals, chemical entities (where the definitions of Chemical Entities and Biopharmaceuticals is that of G. Ashton, 2001, Nature Biotechnology 19, 307-3111)), metabolites, new salts, prodrugs, esters of existing compounds, vaccines, antigens, polyketides, non-ribosomal peptides, vitamins, and molecules from any natural source (such as the plant derived cyclopamine);

(viii) 5-methyl cytosine alterations due to responses at the molecular, cellular, tissue, organ and whole organism levels to RNA and DNA viruses be they single or double stranded, from external sources, or intemally activated such as in endogenous transposons or retrotransposons, (SINES and LINES);

(ix) 5-methyl cytosine alterations due to responses at the molecular, cellular, tissue, organ and whole organism levels to reverse transcribed copies of RNA transcripts be they of genic or non genic origins, (or intron containing or not);

(x) 5-methyl cytosine alterations due to responses at the molecular, cellular, tissue, organ and whole organism levels to: (a) DNA, RNA, PNA, INA, AMA, AHA, etc. (or DNA, RNA, PNA, INA, AMA, AHA, aptamers of any in all combinations); including DNA, RNA, PNA, INA, AMA, AHA, etc molecules circulating in all fluids including blood and cerebrospinal fluid as well as matemal fluids before, during and after pregnancy (b) combinations of conjugated biomolecules that are chimeras of peptides and nucleic acids; or chimeras of natural molecules such as cholesterol moieties, hormones and nucleic acids, and

(xi) 5-methyl cytosine alterations due to responses of stem cells, or cells that have been (or are being) transdetermined or transdifferentiated using a perturbogen in the broadest sense, (including perturbogens from non human sources such as amphibian oocytes, plant, animal, bacterial or viral sources, drugs, antibodies, or any cocktail thereof), along trajectories to any other existing or novel cell type, preferably along the trajectories to or from stem cells, (either carried out in vivo, ex vivo or in association with novel environments or natural and synthetic substrates, or combinations thereof).

Nucleic Acids

The term “nucleic acid” covers the naturally occurring nucleic acids, DNA and RNA. The term “nucleic acid analogues” covers derivatives of the naturally occurring nucleic acids, DNA and RNA, as well as synthetic analogues of naturally occurring nucleic acids. Synthetic analogues comprise one or more nucleotide analogues. The term nucleotide analogue includes all nucleotide analogues capable of being incorporated into a nucleic acid backbone and capable of specific base-pairing (see below), essentially like naturally occurring nucleotides.

Hence the terms “nucleic acid” or “nucleic acid analogues” designate any molecule which essentially consists of a plurality of nucleotides and/or nucleotide analogues and/or intercalator pseudonucleotides. Nucleic acids or nucleic acid analogues useful for the present invention may comprise a number of different nucleotides with different backbone monomer units.

Preferably, single strands of nucleic acids or nucleic acid analogues are capable of hybridising with an substantially complementary single stranded nucleic acid and/or nucleic acid analogue to form a double stranded nucleic acid or nucleic acid analogue. More preferably such a double stranded analogue is capable of forming a double helix. Preferably, the double helix is formed due to hydrogen bonding, more preferably, the ouble helix is a double helix selected from the group consisting of double helices of A orm, B form, Z form and intermediates thereof.

Hence, nucleic acids and nucleic acid analogues useful for the present invention nclude, but is not limited to DNA, RNA, LNA, PNA, MNA, ANA, HNA, INA and mixtures hereof and hybrids thereof, as well as phosphorous atom modifications thereof, such as ut not limited to phosphorothioates, methyl phospholates, phosphoramidites, hosphorodithiates, phosphoroselenoates, phosphotriesters and phosphoboranoates. In addition non-phosphorous containing compounds may be used for linking to nucleotides such as but not limited to methyliminomethyl, formacetate, thioformacetate and linking groups comprising amides. In particular nucleic acids and nucleic acid analogues may comprise one or more intercalator pseudonucleotides.

Within this context “mixture” is meant to cover a nucleic acid or nucleic acid analogue strand comprising different kinds of nucleotides or nucleotide analogues. Furthermore, within this context, “hybrid” is meant to cover nucleic acids or nucleic acid analogues comprising one strand which comprises nucleotide or nucleotide analogue with one or more kinds of backbone and another strands which comprises nucleotide or nucleotide analogue with different kinds of backbone.

By INA is meant an intercalating nucleic acid in accordance with the teaching of WO 03/051901, WO 03/052132, WO 03/052133 and WO 03/052134 (Unest A/S) incorporated herein by reference. By HNA is meant nucleic acids as for example described by Van Aetschot et al., 1995. By MNA is meant nucleic acids as described by Hossain et al, 1998. ANA refers to nucleic acids described by Allert et al, 1999. LNA may be any LNA molecule as described in WO 99/14226 (Exiqon), preferably, LNA is selected from the molecules depicted in the abstract of WO 99/14226. More preferably, LNA is a nucleic acid as described in Singh et al, 1998, Koshkin et al, 1998 or Obika et al., 1997. PNA refers to peptide nucleic acids as for example described by Nielsen et al, 1991.

The term nucleotide designates the building blocks of nucleic acids or nucleic acid analogues and the term nucleotide covers naturally occurring nucleotides and derivatives thereof as well as nucleotides capable of performing essentially the same functions as naturally occurring nucleotides and derivatives thereof. Naturally occurring nucleotides comprise deoxyribonucleotides comprising one of the four main nucleobases adenine (A), thymine (T), guanine (G) or cytosine (C), and ribonucleotides comprising on of the four nucleobases adenine (A), uracil (U), guanine (G) or cytosine (C). In addition to the main or common bases above, other less common naturally occurring bases which can exist in some nucleic acid molecules include 5-methyl cytosine (met-C) and 6-methyl adenine (met-A).

Nucleotide analogues may be any nucleotide like molecule that is capable of being incorporated into a nucleic acid backbone and capable of specific base-pairing. Non-naturally occurring nucleotides includes, but is not limited to the nucleotides omprised within DNA, RNA, PNA, INA, HNA, MNA, ANA, LNA, CNA, CeNA, TNA, (2′-NH)-TNA, (3′-NH)-TNA, α-L-Ribo-LNA, α-L-Xylo-LNA, β-D-Xylo-LNA, α-D-Ribo-LNA, [3.2.1]-LNA, Bicyclo-DNA, 6-Amino-Bicyclo-DNA, 5-epi-Bicyclo-DNA, α-Bicyclo-DNA, Tricyclo-DNA, Bicyclo[4.3.0]-DNA, Bicyclo[3.2.1]-DNA, Bicyclo[4.3.0]amide-DNA, β-D-Ribopyranosyl-NA, α-L-Lyxopyranosyl-NA, 2′-R-RNA, α-L-RNA or α-D-RNA, β-D-RNA.

The function of nucleotides and nucleotide analogues is to be able to interact specifically with complementary nucleotides via hydrogen bonding of the nucleobases of the complementary nucleotides as well as to be able to be incorporated into a nucleic acid or nucleic acid analogue. Naturally occurring nucleotide, as well as some nucleotide analogues are capable of being enzymatically incorporated into a nubleic acid or nucleic acid analogue, for example by RNA or DNA polymerases. However, nucleotides or nucleotide analogues may also be chemically incorporated into a nucleic acid or nucleic acid analogue.

Furthermore nucleic acids or nucleic acid analogues may be prepared by coupling two smaller nucleic acids or nucleic acid analogues to another, for example this may be done enzymatically by ligases or it may be done chemically.

Nucleotides or nucleotide analogues comprise a backbone monomer unit and a nucleobase. The nucleobase may be a naturally occurring nucleobase or a derivative thereof or an analogue thereof capable of performing essentially the same function. The function of a nucleobase is to be capable of associating specifically with one or more other nucleobases via hydrogen bonds. Thus it is an important feature of a nucleobase. that it can only form stable hydrogen bonds with one or a few other nucleobases, but that it can not form stable hydrogen bonds with most other nucleobases usually including itself. The specific interaction of one nucleobase with another nucleobase is generally termed “base-pairing”.

The base pairing results in a specific hybridisation between predetermined and complementary nucleotides. Complementary nucleotides are nucleotides that comprise nucleobases that are capable of base-pairing.

Of the common naturally occurring nucleobases, adenine (A) pairs with thymine (T) or uracil (U); and guanine (G) pairs with cytosine (C). Accordingly, a nucleotide comprising A is complementary to a nucleotide comprising either T or U, and a nucleotide comprising G is complementary to a nucleotide comprising C.

Nucleotides may further be derivatised to comprise an appended molecular entity. The nucleotides can be derivatised on the nucleobases or on the backbone monomer unit. Preferred sites of derivatisation on the bases include the 8-position of adenine, the 5-position of uracil, the 5 or 6-posibon of cytosine, and the 7-position of guanine. The heterocyclic modifications can be grouped into three structural classes: Enhanced base stacking, additional hydrogen bonding, and the combination of these classes. Modifications that enhance base stacking by expanding the π-electron cloud of the planar systems are represented by conjugated, lipophilic modifications in the 5-position of pyrimidines and the 7-position of 7-deaza-purines. Substitutions in the 5-position of pyrimidines modifications include propynes, hexynes, thiazoles and simply a methyl group; and substituents in the 7-position of 7-deaza purines include iodo, propynyl, and cyano groups. It is also possible to modify the 5-position of cytosine from propynes to five-membered heterocycles and to tricyclic fused systems, which emanate from the 4- and 5-position (cytosine clamps). A second type of heterocycle modification is represented by the 2-amino-adenine where the additional amino group provides another hydrogen bond in the A-T base pair, analogous to the three hydrogen bonds in a G-C base pair. Heterocycle modifications providing a combination of effects are represented by 2-amino-7-deaza-7-modified adenine and the tricyclic cytosine analog having an ethoxyamino functional group of heteroduplexes. Furthermore, N2-modified 2-amino adenine modified oligonucleotides are among commonly modifications. Preferred sites of derivatisation on ribose or deoxyribose moieties are modifications of non-connecting carbon positions C-2′ and C-4′, modifications of connecting carbons C-1′, C-3′ and C-5′, replacement of sugar oxygen, O-4′, anhydro sugar modifications (conformational restricted), cyclosugar modifications (conformational restricted), ribofuranosyl ring size change, connection sites—sugar to sugar, (C-3′ to C-5′/C-2′ to C-5′), hetero-atom ring—modified sugars and combinations of above modifications. However, other sites may be derivatised, as long as the overall base pairing specificity of a nucleic acid or nucleic acid analogue is not disrupted. Finally, when the backbone monomer unit comprises a phosphate group, the phosphates of some backbone monomer units may be derivatised.

Oligonucleotide or oligonucleotide analogue as used herein are molecules essentially consisting of a sequence of nucleotides and/or nucleotide analogues and/or intercalator pseudonucleotides. Preferably oligonucleotide or oligonucleotide analogue comprises 5 to 100 individual nucleotides. Oligonucleotide or oligonucleotide analogues may comprise DNA, RNA, LNA, 2′-O-methyl RNA, PNA, ANA, HNA and mixtures thereof, as well as any other nucleotide and/or nucleotide analogue and/or intercalator pseudonucleotide.

Corresponding Nucleic Acids

Nucleic acids, nucleic acid analogues, oligonucleotides or oligonucleotides analogues are considered to be corresponding when they are capable of hybridising. Preferably corresponding nucleic acids, nucleic acid analogues, oligonucleotides or oligonucleotides analogues are capable of hybridising under low stringency conditions, more preferably corresponding nucleic acids, nucleic acid analogues, oligonucleotides or oligonucleotides analogues are capable of hybridising under medium stringency conditions, more preferably corresponding nucleic acids, nucleic acid analogues, oligonucleotides or oligonucleotides analogues are capable of hybridising under high stringency conditions.

High stringency conditions as used herein shall denote stringency as normally applied in connection with Southern blotting and hybridisation as described e.g. by Southern E. M., 1975, J. Mol. Biol. 98:503-517. For such purposes it is routine practise to include steps of prehybridization and hybridization. Such steps are normally performed using solutions containing 6×SSPE, 5% Denhardt's, 0.5% SDS, 50% formamide, 100 μg/ml denatured salmon testis DNA (incubation for 18 hrs at 42° C.), followed by washing with 2×SSC and 0.5% SDS (at room temperature and at 37° C.), and washing with 0.1×SSC and 0.5% SDS (incubation at 68° C. for 30 min), as described by Sambrook et al., 1989, in “Molecular Cloning/A Laboratory Manual”, Cold Spring Harbor), which is incorporated herein by reference.

Medium stringency conditions as used herein shall denote hybridisation in a buffer containing 1 mM EDTA, 10 mM Na₂HPO₄H₂O, 140 mM NaCl, at pH 7.0. Preferably, around 1.5 μM of each nucleic acid or nucleic acid analogue strand is provided. Alternatively medium stringency may denote hybridisation in a buffer containing 50 mM KCl, 10 mM TRIS-HCI (pH 9,0), 0.1% Triton X-100, 2 mM MgCl2.

Low stringency conditions denote hybridisation in a buffer constituting 1 M NaCl, 10 mM Na₃PO₄ at pH 7,0.

Alternatively, corresponding nucleic acids, nucleic acid analogues, oligonucleotides or oligonucleotides, nucleic acid analogues, oligonucleotides or oligonucleotides substantially complementary to each other over a given sequence, such as more than 70% complementary, for example more than 75% complementary, such as more than 80% complementary, for example more than 85% complementary, such as more than 90% complementary, for example more than 92% complementary, such as more than 94% complementary, for example more than 95% complementary, such as more than 96% complementary, for example more than 97% complementary.

Preferably the given sequence is at least 10 nucleotides long, such as at least 15 nucleotides, for example at least 20 nucleotides, such as at least 25 nucleotides, for example at least 30 nucleotides, such as between 10 and 500 nucleotides, for example between 10 and 100 nucleotides long, such as between 10 and 50 nucleotides long. More preferably corresponding oligonucleotides or oligonucleotides analogues are substantially complementary over their entire length.

Cross-Hybridisation

The term cross-hybridisation covers unintended hybridisation between at least two nucleic acids or nucleic acid analogues. Hence the tern cross-hybridization may be used to describe the hybridisation of for example a nucleic acid probe or nucleic acid analogue probe sequence to other nucleic acid sequences or nucleic acid analogue sequences than its intended target sequence.

Often cross-hybridization occurs between a probe and one or more corresponding non-target sequences, even though these have a lower degree of complementarity than the probe and its corresponding target sequence. This unwanted effect could be due to a large excess of probe over target and/or fast annealing kinetics. Cross-hybridization also occurs by hydrogen bonding between few nucleobase pairs, e.g. between primers in a PCR reaction, resulting in primer dimer formation and/ or formation of unspecific PCR products.

Nucleic acids comprising one or more nucleotide analogues with high affinity for nucleotide analogues of the same type tend to form dimer or higher order complexes based on base pairing. Probes comprising nucleotide analogues such as, but not limited to, LNA, 2′-O-methyl RNA and PNA generally have a high affinity for hybridising to other oligonucleotide analogues comprising backbone monomer units of the same type. Hence even though individual probe molecules only have a low degree of complementarity they tend to hybridize.

Self-Hybridisation

The term self-hybridisation covers the process wherein a nucleic acid or nucleic acid analogue molecule anneals to itself by folding back on itself, generating a secondary structure like for example a hairpin structure, or one molecule binding to another identical molecule leading to aggregation of the molecules. In most applications it is of importance to avoid self-hybridization. Furthermore, self hybridzation can also increase background signal and importantly decrease the sensitivity of molecular biological methods or assays. The generation of secondary structures may inhibit hybridisation with desired nucleic acid target sequences. This is undesired in most assays for example when the nucleic acid or nucleic acid analogue is used as primer in PCR reactions or as fluorophore/quencher labelled probe for exonuclease assays. In both assays, self-hybridisation will inhibit hybridization to the target nucleic acid and additionally the degree of fluorophore quenching in the exonuclease assay is lowered.

Nucleic acids comprising one or more nucleotide analogues with high affinity for nucleotide analogues of the same type tend to self-hybridize, Probes comprising nucleotide analogues such as, but not limited to, LNA, 2′-O-methyl RNA and PNA generally have a high affinity for self-hybridising. Hence even though individual probe molecules only have a low degree of self-complementary they tend to self-hybridize.

Melting Temperature

Melting of nucleic acids refer to the separation of the two strands of a double-stranded nucleic acid molecule. The melting temperature (Tₘ) denotes the temperature in degrees celsius at which 50% helical (hybridized) versus coil (unhybridized) forms are present.

A high melting temperature is indicative of a stable complex and accordingly of a high affinity between the individual strands. Similarly, a low melting temperature is indicative of a relatively low affinity between the individual strands. Accordingly, usually strong hydrogen bonding between the two strands results in a high melting temperature.

Furthermore, intercalation of an intercalator between nucleobases of a double stranded nucleic acid may also stabilise double stranded nucleic acids and accordingly result in a higher melting temperature.

In addition, the melting temperature is dependent on the physical/chemical state of the surroundings. For example the melting temperature is dependent on salt concentration and pH.

The melting temperature may be determined by a number of assays, for example it may be determined by using the UV spectrum to determine the formation and breakdown (melting) of hybridisation.

Intercalating nucleic acid (INA) or Intercalator pseudonucleotide Intercalating nucleic acids (INA) are also termed intercalator pseudonucleotides in this specification.

Pseudonucleotides or polynucleotide analogues comprising intercalators and having one or more of the following desirable characteristics:
Intercalate into the double helix at a predetermined position;
I. Substantially increase the affinity for DNA;
II. Inhibit or decrease self and cross hybridisation;
III. Discriminate between different nucleic acids, such as RNA and DNA;
IV. Substantially increase the specificity of hybridisation;
V. Increase nuclease stability;
VI. Enhance strand invasion significantly;
VII. Show a change in fluorescence intensity upon hybridisation.

An intercalator pseudonucleotide has the general structure:
X—Y-Q
wherein

X is a backbone monomer unit capable of being incorporated into the backbone of a nucleic acid or nucleic acid analogue,

Q is an intercalator comprising at least one essentially flat conjugated system, which is capable of co-stacking with nucleobases of DNA; and

Y is a linker moiety linking the backbone monomer unit and the intercalator. More preferably an intercalator pseudonucleotide has the general structure:
X—Y-Q
wherein

X is a backbone monomer unit capable of being incorporated into the backbone of a nucleic acid or nucleic acid analogue of the general formula,

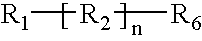

wherein n=1 to 6

R₁ is a trivalent or pentavalent substituted phosphor atom,

R₂ is individually selected from an atom capable of forming at least two bonds, R₂ optionally being individually substituted, and

R₆ is a protecting group;

Q is an intercalator comprising at least one essentially flat conjugated system, which is capable of co-stacking with nucleobases of DNA; and

Y is a linker moiety linking any of R₂ of the backbone monomer unit and the intercalator; and

wherein the total length of Q and Y is in the range from about 7 å to 20 å.

When the intercalator is pyrene, for example, the total length of Q and Y is in the range from about 9 Å to 13 Å, preferably from about 9 Å to 11 Å.

By the term “incorporated into the backbone of a nucleic acid or nucleic acid analogue” is meant that the intercalator pseudonucleotide may be inserted into a sequence of nucleic acids and/or nucleic acid analogues.

By the term “flat conjugated system” is meant that substantially all atoms included in the conjugated system are located in one plane.

By the term “essentially flat conjugated system” is meant that at most 20% of all atoms included in the conjugated system are not located in the one plane at any time.

By the term “conjugated system” is meant a structural unit containing chemical bonds with overlap of atomic p orbitals of three or more adjacent atoms (Gold et al., 1987. Compendium of Chemical Terminology, Blackwell Scientific Publications, Oxford, UK).

Co-stacking is used in short for coaxial stacking. Coaxial stacking is an energetically favorable structure where flat molecules align on top of each other (flat side against flat side) along a common axis in a stack-like structure. Co-stacking requires interaction between two pi-electron clouds of individual molecules in the case of intercalator pseudonucleotides, co-stacking with nucleobases in a duplex, preferably there is an interaction with a pi electron system on an opposite strand, more preferably there is interaction with pi electron systems on both strands. Co-stacking interactions are found both inter- and intra-molecularly. For example nucleic acids adopt a duplex structure to allow nucleobase co-stacking.

Backbone Monomer Unit

Any suitable backbone monomer unit may be employed. The backbone monomer unit comprises the part of an intercalator pseudonucleotide that may be incorporated into the backbone of an oligonucleotide or an oligonucleotide analogue. In addition, the backbone monomer unit may comprise one or more leaving groups, protecting groups and/or reactive groups, which may be removed or changed in any way during synthesis or subsequent to synthesis of an oligonucleotide or oligonucleotide analogue comprising the backbone monomer unit.

The term ‘backbone monomer unit’ only includes the backbone monomer unit per se and it does not include, for example, a linker connecting a backbone monomer unit to an intercalator. Hence, the intercalator as well as the linker is not part of the backbone monomer unit.

Accordingly, backbone monomer units only include atoms, wherein the monomer is incorporated into a sequence, are selected from the group consisting of atoms which are capable of forming a linkage to the backbone monomer unit of a neighboring nucleotide; or

atoms which at least at two sites are connected to other atoms of the backbone monomer unit; or

atoms which at one site is connected to the backbone monomer unit and otherwise is not connected with other atoms.

Backbone monomer unit atoms are thus defined as the atoms involved in the direct linkage (shortest path) between the backbone Phosphor-atoms of neighbouring nucleotides, when the monomer is incorporated into a sequence, wherein the neighbouring nucleotides are naturally occurring nucleotides.

The backbone monomer unit may be any suitable backbone monomer unit. The backbone monomer unit may for example be selected-from the group consisting of the backbone monomer units of DNA, RNA, PNA, INA, HNA, MNA, ANA, LNA, CNA, CeNA, TNA, (2′-NH)-TNA. (3′-NH)-TNA, α-L-Ribo-LNA, α-L-Xylo-LNA, β-D-Xylo-LNA, α-D-Ribo-LNA, [3.2.1]-LNA, Bicyclo-DNA, 6-Amino-Bicyclo-DNA, 5-epi-Bicyclo-DNA, α-Bicyclo-DNA, Tricyclo-DNA, Bicyclo[4.3.0]-DNA, Bicyclo[3.2.1]-DNA, Bicyclo[4.3.0]amide-DNA, β-D-Ribopyranosyl-NA, α-L-Lyxopyranosyl-NA, 2′-R-RNA, α-L-RNA or α-D-RNA, β-D-RNA.

The backbone monomer unit of LNA (locked nucleic acid) is a sterically restricted DNA backbone monomer unit, which comprises an intramolecular bridge that restricts the usual conformational freedom of a DNA backbone monomer unit. LNA may be any LNA molecule as described in WO99/14226 (Exiqon). Preferred LNA comprises a methyl linker connecting the 2′-O position to the 4′-C position, however other LNA's such as LNA's wherein the 2′ oxy atom is replaced by either nitrogen or sulphur are also comprised within the present invention.

The backbone monomer unit of intercalator pseudonucleotides preferably have the general structure before being incorporated into an oligonucleotide and/or nucleotide analogue: 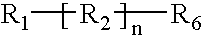
wherein

n=1 to 6, preferably n=2 to 6, more preferably n=3 to 6, more preferably n=2 to 5, more preferably n=3 to 5, more preferably n=3 to 4;

R₁ is a trivalent or pentavalent substituted phosphor atom, preferably R₁ is 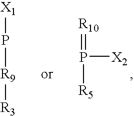
wherein

R₂ may individually be selected from an atom capable of forming at least two bonds, the atom optionally being individually substituted, preferably R₂ is individually selected from O, S, N, C, P, optionally individually substituted. By the term “individually” is meant that R₂ can represent one, two or more different groups in the same molecule. The bonds between two R₂ may be saturated or unsaturated or a part of a ring system or a combination thereof. Each R₂ may individually be substituted with any suitable substituent, such as a substituent selected from H, lower alkyl, C2-C6 alkenyl, C6-C10 aryl, C7-C11 arylmethyl, C2-C7 acyloxymethyl, C3-C8 alkoxycarbonyloxymethyl, C7-C11 aryloyloxymethyl, C3-C8 S-acyl-2-thioethyl.

An “alkyl” group refers to an optionally substituted saturated aliphatic hydrocarbon, including straight-chain, branched-chain, and cyclic alkyl groups. Preferably, the alkyl group has 1 to 25 carbons and contains no more than 20 heteroatoms. More preferably, it is a lower alkyl of from 1 to 12 carbons, more preferably 1 to 6 carbons, more preferably 1 to 4 carbons. Heteroatoms are preferably selected from the group consisting of nitrogen, sulfur, phosphorus, and oxygen.

An “alkenyl” group refers to an optionally substituted hydrocarbon containing at least one double bond, including straight-chain, branched-chain, and cyclic alkenyl groups, all of which may be optionally substituted. Preferably, the alkenyl group has 2 to 25 carbons and contains no more than 20 heteroatoms. More preferably, it is a lower alkenyl of from 2 to 12 carbons, more preferably 2 to 4 carbons. Heteroatoms are preferably selected from the group consisting of nitrogen, sulfur, phosphorus, and oxygen.

An “alkynyl” group refers to an optionally substituted unsaturated hydrocarbon containing at least one triple bond, including straight-chain, branched-chain, and cyclic alkynyl groups, all of which may be optionally substituted. Preferably, the alkynyl group has 2 to 25 carbons and contains no more than 20 heteroatoms. More preferably, it is a lower alkynyl of from 2 to 12 carbons, more preferably 2 to 4 carbons. Heteroatoms are preferably selected from the group consisting of nitrogen, sulfur, phosphorus, and oxygen.

An “aryl” refers to an optionally substituted aromatic group having at least one ring with a conjugated pi electron system and includes carbocyclic aryl, heterocyclic aryl, bi-aryl, and tri-aryl groups. Examples of aryl substitution substituents include alkyl, alkenyl, alkynyl, aryl, amino, substituted amino, carboxy, hydroxy, alkoxy, nitro, sulfonyl, halogen, thiol and aryloxy.

A “carbocyclic aryl” refers to an aryl where all the atoms on the aromatic ring are carbon atoms. The carbon atoms are optionally substituted as described above for an aryl. Preferably, the carbocyclic aryl is an optionally substituted phenyl.

A “heterocyclic aryl” refers to an aryl having 1 to 3 heteroatoms as ring atoms in the aromatic ring and the remainder of the ring atoms are carbon atoms. Suitable heteroatoms include oxygen, sulfur, and nitrogen. Examples of heterocyclic aryls include furanyl, thienyl, pyridyl, pyrrolyl, N-lower alkyl pyrrolo, pyrimidyl, pyrazinyl, and imidazolyl. The heterocyclic aryl is optionally substituted as described above for an aryl.

The substituents on two or more R₂ may alternatively join to form a ring system, such as any of the ring systems as defined above. Preferably R₂ is substituted with an atom or a group selected from H, methyl, R₄, hydroxyl, halogen, and amino, more preferably R₂ is substituted with an atom or a group selected from H, methyl, R₄. More preferably R₂ is individually selected from O, S, NH, N(Me), N(R₄), C(R₄)₂, CH(R₄) or CH₂, wherein R₄ is as defined below.

R₃ is methyl, beta-cyanoethyl, p-nitrophenetyl, o-chlorophenyl, or p-chlorophenyl.

R₄ is lower alkyl, preferably lower alkyl such as methyl, ethyl, or isopropyl, or heterocyclic, such as morpholino, pyrrolidino, or 2,2,6,6-tetramethylpyrrolidino, wherein lower alkyl is defined as C₁-C₈, such as C₁-C₄.

R₅ is alkyl, alkoxy, aryl or H, with the proviso that R₅ is H when X₂═O⁻, preferably R₅ is selected from lower alkyl, lower alkoxy, aryloxy. In a preferred embodiment aryloxy is selected from phenyl, naphtyl or pyridine.

R₆ is a protecting group, selected from any suitable protecting groups. Preferably R₆ is selected from the group consisting of trityl, monomethoxytrityl, 2-chlorotrityl, 1,1,1,2-tetrachloro-2,2-bis(p-methoxyphenyl)-ethan (DATE), 9-phenylxanthine-9-yl (pixyl) and 9-(p-methoxyphenyl) xanthine-9-yl (MOX) or other protecting groups mentioned in “Current Protocols In Nucleic Acid Chemistry” volume 1, Beaucage et al. Wiley. More preferably, the protecting group may be selected from the group consisting of monomethoxytrityl and dimethoxytrityl. Most preferably, the protecting group may be 4,4′-dimethoxytrityl (DMT).

R₉ is selected from O, S, N optionally substituted, preferably R₉ is selected from O, S, NH, N(Me).

R₁₀ is selected from O, S, N, C, optionally substituted.

X₁ is selected from Cl, Br, I, or N(R₄)₂

X₂ is selected from Cl, Br, I, N(R₄)₂, or O⁻

As described above with respect to the substituents the backbone monomer unit an be acyclic or part of a ring system.

Preferably, the backbone monomer unit of an intercalator pseudonucleotide is selected from the group consisting of acyclic backbone monomer units. Acyclic is meant to cover any backbone monomer unit, which does not comprise a ring structure, for example the backbone monomer unit preferably does not comprise a ribose or a deoxyribose group.

In particular, it is preferred that the backbone monomer unit of an intercalator pseudonucleotide is an acyclic backbone monomer unit, which is capable of stabilising a bulge insertion (defined below).

The backbone monomer unit of an intercalator pseudonucleotide may be selected from the group consisting of backbone monomer units comprising at least one chemical group selected from trivalent and pentavalent phosphorous atom such as a pentavalent phosphorous atom. More preferably, the phosphate atom of the backbone monomer unit of an intercalator pseudonucleotide may be selected from the group consisting of backbone monomer units comprising at least one chemical group selected from the group consisting of, phosphoester, phosphodiester, phosphoramidate and phosphoramidite groups.

Preferred backbone monomer units comprising at least one chemical group selected from the group consisting of phosphate, phosphoester, phosphodiester, phosphoramidate and phosphoramidite groups are backbone monomer units, wherein the distance from at least one phosphor atom to at least one phosphor atom of a neighbouring nucleotide, not including the phosphor atoms, is at the most 6 atoms long, for example 2, such as 3, for example 4, such as 5, for example 6 atoms long, when the backbone monomer unit is incorporated into a nucleic acid backbone.

Preferably, the backbone monomer unit is capable of being incorporated into a phosphate backbone of a nucleic acid or nucleic acid analogue in a manner so that at the most 5 atoms (more preferably at most 4) are separating the phosphor atom of the intercalator pseudonucleotide backbone monomer unit and the nearest neighbouring phosphor atom, more preferably 5 atoms are separating the phosphor atom of the intercalator pseudonucleotide backbone monomer unit and the nearest neighbouring phosphor atom, in both cases not including the phosphor atoms themselves.

In a particularly preferred form, the intercalator pseudonucleotide comprises a backbone monomer unit that comprises a phosphoramidite and more preferably the backbone monomer unit comprises a trivalent phosphoramidite. Suitable trivalent phosphoramidites are trivalent phosphoramidites that may be incorporated into the backbone of a nucleic acid and/or a nucleic acid analogue. Usually, the amidit group may not be incorporated into the backbone of a nucleic acid, but rather the amidit group or part of the amidit group may serve as a leaving group and/or protecting group. However, it is preferred that the backbone monomer unit comprises a phosphoramidite group because such a group may facilitate the incorporation of the backbone monomer unit into a nucleic acid backbone.

The backbone monomer unit of an intercalator pseudonucleotide which is inserted into an oligonucleotide or oligonucleotide analogue, may comprise a phosphodiester bond. Additionally, the backbone monomer unit of an intercalator pseudonucleotide may comprise a pentavalent phosphoramidate. Preferably, the backbone monomer unit of an intercalator pseudonucleotide is an acyclic backbone monomer unit that may comprise a pentavalent phosphoramidate.

Leaving Group

The backbone monomer unit may comprise one or more leaving groups. Leaving groups are chemical groups, which are part of the backbone monomer unit when the intercalator pseudonucleotide or the nucleotide is a monomer, but which are no longer present in the molecule once the intercalator pseudonucleotide or the nucleotide has been incorporated into an oligonucleotide or oligonucleotide analogue.

The nature of a leaving group depends of the backbone monomer unit. For example, when the backbone monomer unit is a phosphor amidit, the leaving group may, for example be an diisopropylamine group. In general, when the backbone monomer unit is a phosphor amidit, a leaving group is attached to the phosphor atom for example in the form of diisopropylamine and the leaving group is removed upon coupling of the phosphor atom to a nucleophilic group, whereas the rest of the phosphate group or part of the rest, may become part of the nucleic acid or nucleic acid analogue backbone.

Reactive Group

The backbone monomer units may furthermore comprise a reactive group which is capable of performing a chemical reaction with another nucleotide or oligonucleotide or nucleic acid or nucleic acid analogue to form a nucleic acid or nucleic acid analogue, which is one nucleotide longer than before the reaction. Accordingly, when nucleotides are in their free form, i.e. not incorporated into a nucleic acid, they may comprise a reactive group capable of reacting with another nucleotide or a nucleic acid or nucleic acid analogue.

The reactive group may be protected by a protecting group. Prior to the chemical reaction, the protection group may be removed. The protection group will thus not be a part of the newly formed nucleic acid or nucleic acid analogue. Examples of reactive groups are nucleophiles such as the 5′-hydroxy group of DNA or RNA backbone monomer units.

Protecting Group

The backbone monomer unit may also comprise a protecting group which can be removed during synthesis. Removal of the protecting group allows for a chemical reaction between the intercalator pseudonucleotide and a nucleotide or nucleotide analogue or another intercalator pseudonucleotide.

In particular, a nucleotide monomer or nucleotide analogue monomer or intercalator pseudonucleotide monomer may comprise a protecting group, which is no longer present in the molecule once the nucleotide or nucleotide analogue or intercalator pseudonucleotide has been incorporated into a nucleic acid or nucleic acid analogue. Furthermore, backbone monomer units may comprise protecting groups which may be present in the oligonucleotide or oligonucleotide analogue subsequent to incorporation of the nucleotide or nucleotide analogue or intercalator pseudonucleotide, but which may no longer be present after introduction of an additional nucleotide or nucleotide analogue to the oligonucleotide or oligonucleotide analogue or which may be removed after the synthesis of the entire oligonucleotide or oligonucleotide analogue.

The protecting group may be removed by a number of suitable techniques known to the person skilled in the art. Preferably, the protecting group may be removed by a treatment selected from the group consisting of acid treatment, thiophenol treatment and alkali treatment.

Preferred protecting groups, which may be used to protect the 5′ end or the 5′ end analogue of a backbone monomer unit may be selected from the group consisting of trityl, monomethoxytrityl, 2-chlorotrityl, 1,1,1,2-tetrachloro-2,2-bis(p-methoxyphenyl)-ethan (DATE), 9-phenylxanthine-9-yl (pixyl) and 9-(p-methoxyphenyl) xanthine-9-yl (MOX) or other protecting groups mentioned in “Current Protocols In Nucleic Acid Chemistry” volume 1, Beaucage et al. Wiley. More preferably the protecting group may be selected from the group consisting of monomethoxytrityl and dimethoxytrityl. Most preferably, the protecting group may be 4,4′-dimethoxytrityl(DMT). 4,4′-dimethoxytrityl(DMT) groups may be removed by acid treatment, for example by brief incubation (30 to 60 seconds sufficient) in 3% trichloroacetic acid or in 3% dichloroacetic acid in CH₂Cl₂.

Preferred protecting groups which may protect a phosphate or phosphoramidite group of a backbone monomer unit may for example be selected from the group consisting of methyl and 2-cyanoethyl. Methyl protecting groups may for example be removed by treatment with thiophenol or disodium 2-carbamoyl 2-cyanoethylene-1,1-dithiolate. 2-cyanoethyl-groups may be removed by alkali treatment, for example treatment with concentrated aqueous ammonia, a 1:1 mixture of aqueous methylamine and concentrated aqueous ammonia or with ammonia gas.

Intercalator

The term intercalator covers any molecular moiety comprising at least one essentially flat conjugated system, which is capable of co-stacking with nucleobases of a nucleic acid. Preferably an intercalator consists of at least one essentially flat conjugated system which is capable of co-stacking with nucleobases of a nucleic acid or nucleic acid analogue.

Preferably, the intercalator comprises a chemical group selected from the group consisting of polyaromates and heteropolyaromates an even more preferably the intercalator essentially consists of a polyaromate or a heteropolyaromate. Most preferably, the intercalator is selected from the group consisting of polyaromates and heteropolyaromates.

Polyaromates or heteropolyaromates may consist of any suitable number of rings, such as 1, for example 2, such as 3, for example 4, such as 5, for example 6, such as 7, for example 8, such as more than 8. Furthermore polyaromates or heteropolyaromates may be substituted with one or more selected from the group consisting of hydroxyl, bromo, fluoro, chloro, iodo, mercapto, thio, cyano, alkylthio, heterocycle, aryl, heteroaryl, carboxyl, carboalkoyl, alkyl, alkenyl, alkynyl, nitro, amino, alkoxyl and amido.

In one preferred form, the intercalator may be selected from the group consisting of polyaromates and heteropolyaromates that are capable of fluorescing.

In another more preferred form, the intercalator may be selected from the group consisting of polyaromates and heteropolyaromates that are capable of forming excimers, exciplexes, fluorescence resonance energy transfer (FRET) or charged transfer complexes.

Accordingly, the intercalator may preferably be selected from the group consisting of phenanthroline, phenazine, phenanthridine, anthraquinone, pyrene, anthracene, napthene, phenanthrene, picene, chrysene, naphtacene, acridones, benzanthracenes, stilbenes, oxalo-pyridocarbazoles, azidobenzenes, porphyrins, psoralens and any of the aforementioned intercalators substituted with one or more selected from the group consisting of hydroxyl, bromo, fluoro, chloro, iodo, mercapto, thio, cyano, alkylthio, heterocycle, aryl, heteroaryl, carboxyl, carboalkoyl, alkyl, alkenyl, alkynyl, nitro, amino, alkoxyl and/or amido.

Preferably, the intercalator is selected from the group-consisting of phenanthroline, phenazine, phenanthridine, anthraquinone, pyrene, anthracene, napthene, phenanthrene, picene, chrysene, naphtacene, acridones, benzanthracenes, stilbenes, oxalo-pyridocarbazoles, azidobenzenes, porphyrins and psoralens.

The examples of intercalators are not to be understood as limiting in any way, but only as to provide examples of possible structures for use as intercalators. In addition, the substitution of one or more chemical groups on each intercalator to obtain modified structures is also included.

The intercalator moiety of the intercalator pseudonucleotide is linked to the backbone unit by the linker. When going from the backbone along the linker to the intercalating moiety, the linker and intercalator connection is defined as the bond between a linker atom and the first atom being part of a conjugated system that is able to co-stack with nucleobases of a strand of a oligonucleotide or oligonucleotide analogue when the oligonucleotide or oligonucleotide analogue is hybridized to an oligonucleotide analogue comprising the intercalator pseudonucleotide.

The linker may comprise a conjugated system and the intercalator may comprise another conjugated system. In this case the linker conjugated system is not capable of co-stacking with nucleobases of the opposite oligonucleotide or oligonucleotide analogue strand.

Linker

The linker of a intercalator pseudonucleotide is a moiety connecting the intercalator and the backbone monomer of the intercalator pseudonucleotide. The linker may comprise one or more atom(s) or bond(s) between atoms.

By the definitions of backbone and intercalating moieties defined herein, the linker is the shortest path linking the backbone and the intercalator. If the intercalator is linked directly to the backbone, the linker is a bond. The linker usually consists of a chain of atoms or a branched chain of atoms. Chains can be saturated as well as unsaturated. The linker may also be a ring structure with or without conjugated bonds. For example, the linker may comprise a chain of m atoms selected from the group consisting of C, O, S, N, P, Se, Si, Ge, Sn and Pb, wherein one end of the chain is connected to the intercalator and the other end of the chain is connected to the backbone monomer unit.

The total length of the linker and the intercalator of the intercalator pseudonucleotides preferably is between 8 and 13 Å. Accordingly, m should be selected dependent on the size of the intercalator of the specific intercalator pseudonucleotide. That is, m should be relatively large, when the intercalator is small and m should be relatively small when the intercalator is large. For most purposes, however, m will be an integer from 1 to 7, such as from 1 to 6, such as from 1 to 5, such as from 1 to 4. As described above, the linker may be an unsaturated chain or another system involving conjugated bonds. For example, the linker may comprise cyclic conjugated structures. Preferably, m is from 1 to 4 when the linker is an saturated chain.

When the intercalator is pyrene, m is preferably an integer from 1 to 7, such as from 1 to 6, such as from 1 to 5, such as from 1 to 4, more preferably from 1 to 4, even more preferably from 1 to 3, most preferably m is 2 or 3.

When the intercalator has the structure

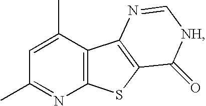

m is preferably from 2 to 6, more preferably 2.

The chain of the linker may be substituted with one or more atoms selected from the group consisting of C, H, O, S, N, P, Se, Si, Ge, Sn and Pb.

In one form, the linker is an azaalkyl, oxaalkyl, thiaalkyl or alkyl chain. For example, the linker may be an alkyl chain substituted with one or more selected from the group consisting C, H, O, S, N, P, Se, Si, Ge, Sn and Pb. In a preferred embodiment the linker consists of an unbranched alkyl chain, wherein one end of the chain is connected to the intercalator and the other end of the chain is connected to the backbone monomer unit and wherein each C is substituted with 2 H. More preferably, the unbranched alkyl chain is from 1 to 5 atoms long, such as from 1 to 4 atoms long, such as from 1 to 3 atoms long, such as from 2 to 3 atoms long.

In another form, the linker is a ring structure comprising atoms selected from the group consisting of C, O, S, N, P, Se, Si, Ge, Sn and Pb. For example the linker may be such a ring structure substituted with one or more selected from the group consisting of C, H, O, S, N, P, Se, Si, Ge, Sn and Pb.

In another form, the linker consists of from 1 to −6 C atoms, from 0 to 3 of each of the following atoms O, S, N. More preferably the linker consists of from 1 to 6 C atoms and from 0 to 1 of each of the atoms O, S, N. In a preferred form, the linker consists of a chain of C, O, S and N atoms, optionally substituted. Preferably the chain should consist of at the most 3 atoms, thus comprising from 0 to 3 atoms selected individually from C, O, S, N, optionally substituted.

In a preferred form, the linker consists of a chain of C, N, S and O atoms, wherein one end of the chain is connected to the intercalator and the other end of the chain is connected to the backbone monomer unit.

The linker constitutes Y in the formula for the intercalator pseudonucleotide X—Y-Q, as defined above, and hence X and Q are not part of the linker.

Intercalator Pseudonucleotides

Intercalator pseudonucleotides or INA molecules preferably have the general structure
X—Y-Q
wherein

X is a backbone monomer unit capable of being incorporated into the backbone of a nucleic acid or nucleic acid analogue;

Q is an intercalator comprising at least one essentially flat conjugated system, which is capable of co-stacking with nucleobases of a nucleic acid; and

Y is a linker moiety linking the backbone monomer unit and the intercalator; herein the total length of Q and Y is in the range from about 7 Å to 20 Å.

Furthermore, in a preferred embodiment of the present invention the intercalator pseudonucleotide comprises a backbone monomer unit, wherein the backbone monomer unit is capable of being incorporated into the phosphate backbone of a nucleic acid or nucleic acid analogue in a manner so that at the most 4 atoms are separating the two phosphor atoms of the backbone that are closest to the intercalator.

The intercalator pseudonucleotides preferably do not comprise a nucleobase capable of forming Watson-Crick hydrogen bonding. Hence intercalator pseudonucleotides are preferably not capable of Watson-Crick base pairing.

Preferably, the total length of Q and Y is in the range from about 7 Å to 20 Å, more preferably, from about 8 Å to 15 Å, even more preferably from about 8 Å to 13 Å, even more preferably from about 8.4 Å to 12 Å, most preferably from about 8.59 Å to 10 Å or from about 8.4 Å to 10.5 Å.

When the intercalator is pyrene for example, the total length of Q and Y is preferably in the range of about 8 Å to 13 Å, such as from about 9 Å to 13 Å, more preferably from about 9.05 Å to 11 Å, such as from about 9.0 Å to 11 Å, even more preferably from about 9.05 to 10 Å, such as from about 9.0 to 10 Å, most preferably about 9.8 Å.

The total length of the linker (Y) and the intercalator (Q) should be determined by determining the distance from the center of the non-hydrogen atom of the linker which is furthest away from the intercalator to the center of the non-hydrogen atom of the essentially flat, conjugated system of the intercalator that is furthest away from the backbone monomer unit. Preferably, the distance should be the maximal distance in which bonding angles and normal chemical laws are not broken or distorted in any way.

The distance should preferably be determined by calculating the structure of the free intercalating pseudonucleotide with the lowest conformational energy level, and then determining the maximum distance that is possible from the center of the non-hydrogen atom of the linker which is furthest away from the intercalator to the center of the non-hydrogen atom of the essentially flat, conjugated system of the intercalator that is furthest away from the backbone monomer unit without bending, stretching or otherwise distorting the structure more than simple rotation of bonds that are free to rotate (e.g. not double bonds or bonds participating in a ring structure). Preferably the energetically favorable structure is found by ab initio or force fields calculations.

The distance can be determined by a method consisting of the following steps:

the structure of the intercalator pseudonucleotide of interest is drawn by computer using the programme ChemWindow® 6.0 (BioRad);

the structure is transferred to the computer programme SymApps™ (BioRad);

the 3-dimensional structure comprising calculated lengths of bonds and bonding angles of the intercalator pseudonucleotide is calculated using the computer programme SymApps™ (BioRad);

the 3 dimensional structure is transferred to the computer programme RasWin Molecular Graphics Ver. 2.6-ucb;

the bonds are rotated using RasWin Molecular Graphics Ver. 2.6-ucb to obtain the maximal distance (the distance as defined herein above); and

the distance is determined.

Intercalator pseudonucleotides may be any combination of the above mentioned backbone monomer units, linkers and intercalators.

In another preferred form, the intercalator pseudonucleotide is selected from the group consisting of phosphoramidites of 1-(4,4′-dimethoxytriphenylmethyloxy)-3-pyrenemethyloxy-2-propanol. Even more preferably, the intercalator pseudonucleotide is selected from the group consisting of the phosphoramidite of (S)-1-(4,4′-dimethoxytriphenylmethyloxy)-3-pyrenemethyloxy-2-propanol and the phosphoramidite of (R)-1-(4,4′-dimethoxytriphenylmethyloxy)-3-pyrenemethyloxy-2-propanol.

Preparation of Intercalator Pseudonucleotides

The intercalator pseudonucleotides or INA molecules may be synthesised by any suitable method. One suitable method comprises the steps of
a1) providing a compound containing an intercalator comprising at least one essentially flat conjugated system, which is capable of co-stacking with nucleobases of a nucleic acid and optionally a linker part coupled to a reactive group;
b1) providing a linker precursor molecule comprising at least two reactive groups, the two reactive groups may optionally be individually protected; and
c1) reacting the intercalator with the linker precursor and thereby obtaining an intercalator-linker;
d1) providing a backbone monomer precursor unit comprising at least two reactive groups, the two reactive groups may optionally be individually protected and/or masked and optionally comprising a linker part; and
e1) reacting the intercalator-linker with the backbone monomer precursor and obtaining an intercalator-linker-backbone monomer precursor; or
a2) providing a backbone monomer precursor unit comprising at least two reactive groups, the two reactive groups may optionally be individually protected and/or masked and optionally comprising a linker part;
b2) providing a linker precursor molecule comprising at least two reactive groups, the two reactive groups may optionally be individually protected;
c2) reacting the monomer precursor unit with the linker precursor and thereby obtaining a backbone-linker;
d2) providing a compound containing an intercalator comprising at least one essentially flat conjugated system, which is capable of co-stacking with nucleobases of a nucleic acid and optionally a linker part coupled to a reactive group; and
e2) reacting the intercalator with the backbone-linker and obtaining an intercalator-linker-backbone monomer precursor; or
a3) providing a compound containing an intercalator comprising at least one essentially flat conjugated system, which is capable of co-stacking with nucleobases of a nucleic acid and a linker part coupled to a reactive group;
b3) providing a backbone monomer precursor unit comprising at least two reactive groups, the two reactive groups may optionally be individually protected and/or masked), and a linker part;
c3) reacting the intercalator-linker part with the backbone monomer precursor-linker and obtaining an intercalator-linker-backbone monomer precursor;
f) optionally protecting and/or de-protecting the intercalator-linker-backbone monomer precursor;
g) providing a phosphor containing compound capable of linking two psuedonucleotides, nucleotides and/ or nucleotide analogues together;
h) reacting the phosphorous containing compound with the intercalator-linker-backbone monomer precursor; and
i) obtaining an intercalator pseudonucleotide.

Preferably, the intercalator reactive group is selected so that it may react with the linker reactive group. Hence, if the linker reactive group is a nucleophil, then preferably the intercalator reactive group is an electrophile, more preferably an electrophile selected from the group consisting of halo alkyl, mesyloxy alkyl and tosyloxy alkyl. More preferably the intercalator reactive group is chloromethyl. Alternatively, the intercalator reactive group may be a nucleophile group for example a nucleophile group comprising hydroxy, thiol, selam, amine or mixture thereof.

Preferably, the cyclic or non cyclic alkane may be a poly-substituted alkane or alkoxy comprising at least three linker reactive groups. More preferably the poly-substituted alkane may comprise three nucleophilic groups such as, but not limited to, an alkane triole, an aminoalkane diol or mercaptoalkane diol. Preferably the poly-substituted alkane contain one nucleophilic group that is more reactive than the others, alternatively two of the nucleophilic groups may be protected by a protecting group. More preferably the cyclic or non cyclic alkane is 2,2-dimethyl-4-methylhydroxy-1,3-dioxalan, even more preferably the alkane is D-α,β-isopropylidene glycerol.

Preferably, the linker reactive groups should be able to react with the intercalator reactive groups, for example the linker reactive groups may be a nucleophile group for example selected from the group consisting of hydroxy, thiol, selam and amine, preferably a hydroxy group. Alternatively the linker reactive group may be an electrophile group, for example selected from the group consisting of halogen, triflates, mesylates and tosylates. In a preferred form, at least 2 linker reactive groups may be protected by a protecting group.

The method may further comprise a step of attaching a protecting group to one or more reactive groups of the intercalator-precursor monomer. For example a DMT group may be added by providing a DMT coupled to a halogen, such as Cl, and reacting the DMT-Cl with at least one linker reactive group. Accordingly, preferably at least one linker reactive group will be available and one protected. If this step is done prior to reaction with the phosphor comprising agent, then the phosphor comprising agent may only interact with one linker reactive group.

The phosphor comprising agent may for example be a phosphoramidite, for example NC(CH₂)₂OP(Np¹₂)₂ or NC(CH₂)₂OP(NP¹₂)Cl. Preferably the phosphor comprising agent may be reacted with the intercalator-precursor in the presence of a base, such as N(et)₃, N(′pr)₂Et and CH₂Cl₂.

One specific example of a method of synthesising an intercalator pseudonucleotide is outlined in example 1 and in FIG. 7.

Once the appropriate sequences of oligonucleotide or oligonucleotide analogue are determined, they are preferably chemically synthesised using commercially available methods and equipment. For example, the solid phase phosphoramiditee method can be used to produce short oligonucleotide or oligonucleotide analogue comprising intercalator pseudonucleotides.

For example the oligonucleotides or oligonucleotide analogues may be synthesised by any of the methods described in “Current Protocols in Nucleic acid Chemistry” Volume 1, Beaucage et al., Wiley.

Oligonucleotides Comprising Intercalator Pseudonucleotides

High affinity of synthetic nucleic acids towards target nucleic acids may greatly facilitate detection assays and furthermore synthetic nucleic acids with high affinity towards target nucleic acids may be useful for a number of other purposes, such as gene targeting and purification of nucleic acids. Oligonucleotides or oligonucleotide analogues comprising intercalators have been shown to increase affinity for homologous complementary nucleic acids.

Oligonucleotides or oligonucleotide analogues comprising at least one intercalator pseudonucleotide can be made wherein the melting temperature of a hybrid consisting of the oligonucleotides or oligonucleotide analogues and a homologous complementary DNA (DNA hybrid) is significantly higher than the melting temperature of a hybrid between an oligonucleotide or oligonucleotide analogue lacking intercalator pseudonucleotide(s) consisting of the same nucleotide sequence as the oligonucleotide or oligonucleotide analogue and the homologous complementary DNA (corresponding DNA hybrid).

Preferably, the melting temperature of the DNA hybrid is from 1 to 80° C., more preferably at least 2° C., even more preferably at least 5° C., yet more preferably at least 10° C. higher than the melting temperature of the corresponding DNA hybrid.

Oligonucleotides or oligonucleotide analogues can have at least one internal intercalator pseudonucleotide. Positioning intercalator units internally allows for greater flexibility in design. Nucleic acid analogues comprising internally positioned intercalator pseudonucleotides may thus have higher affinity for homologous complementary nucleic acids than nucleic acid analogues that does not have internally positioned intercalator pseudonucleotides. Oligonucleotides or Oligonucleotide analogues comprising at least one internal intercalator pseudonucleotide may also be able to discriminate between RNA (including RNA-like nucleic acid analogues) and DNA (including DNA-like nucleic acid analogues). Furthermore internally positioned fluorescent intercalator monomers could find use in diagnostic tools.

The intercalator pseudonucleotides may be placed in any desirable position within a given oligonucleotide or oligonucleotide analogue. For example, an intercalator pseudonucleotide may be placed at the end of the oligonucleotide or oligonucleotide analogue or an intercalator pseudonucleotide may be placed in an internal position within the oligonucleotide or oligonucleotide analogue.

When the oligonucleotide or oligonucleotide analogue comprise more than 1 intercalator pseudonucleotide, the intercalator pseudonucleotides may be placed in any position in relation to each other. For example they may be placed next to each other, or they may be positioned so that 1, such as 2, for example 3, such as 4, for example 5, such as more than 5 nucleotides are separating the intercalator pseudonucleotides. In one preferred embodiment two intercalator pseudonucleotides within an oligonucleotide or oligonucleotide analogue are placed as next nearest neighbours, i.e. they can be placed at any position within the oligonucleotide or oligonucleotide analogue and having 1 nucleotide separating the two intercalator pseudonucleotides. In another preferred form, two intercalators are placed at or in close proximity to each end respectively of the oligonucleotide or oligonucleotide analogue.

The oligonucleotides or oligonucleotide analogues may comprise any kind of nucleotides and/or nucleotide analogues, such as the nucleotides and/or nucleotide analogues described herein above. For example, the oligonucleotides or oligonucleotide analogues may comprise nucleotides and/or nucleotide analogues comprised within DNA, RNA, LNA, PNA, ANA INA, and HNA. Accordingly, the oligonucleotides or oligonucleotide analogue may comprise one or more selected from the group consisting of subunits of PNA, Homo-DNA, b-D-Altropyranosyl-NA, b-D-Glucopyranosyl-NA, b-D-Allopyranusyl-NA, HNA, MNA, ANA, LNA, CNA, CeNA, TNA, (2′-NH)-TNA, (3′-NH)-TNA, □-L-Ribo-LNA, □-L-Xylo-LNA, □-D-Xylo-LNA, □-D-Ribo-LNA, [3.2.1]-LNA, Bicyclo-DNA, 6-Amino-Bicyclo-DNA, 5-epi-Bicyclo-DNA, □-Bicyclo-DNA, Tricyclo-DNA, Bicyclo[4.3,0]-DNA, Bicyclo[3.2.1]-DNA, Bicyclo[4.3.0]amide-DNA, O-D-Ribopyranosyl-NA, □-L-Lyxopyranosyl-NA, 2′-R-RNA, 2′-OR-RNA, □-L-RNA, α-D-RNA, β-D-RNA, i.e. the oligonucleotide analogue may be selected from the group of PNA, Homo-DNA, b-D-Altropyranosyl-NA, b-D-Glucopyranosyl-NA, b-D-Allopyranusyl-NA, HNA, MNA, ANA, LNA, CNA, CeNA, TNA, (2′-NH)-TNA, (3′-NH)-TNA, □-L-Ribo-LNA, □-L-Xylo-LNA, □-D-Xylo-LNA, □-D-Ribo-LNA, [3.2.1]-LNA, Bicyclo-DNA, 6-Amino-Bicyclo-DNA, 5-epi-Bicyclo-DNA, O-Bicyclo-DNA, Tricyclo-DNA, Bicyclo[4.3.0]-DNA, Bicyclo[3.2.1]-DNA, Bicyclo[4.3.0]amide-DNA, □-D-Ribopyranosyl-NA, □-L-Lyxopyranosyl-NA, 2′-R-RNA, 2′-OR-RNA, □-L-RNA, α-D-RNA, β-D-RNA and mixtures thereof.

One advantage of the oligonucleotides or oligonucleotide analogues is that the melting temperature of a hybrid consisting of an oligonucleotide or oligonucleotide analogue comprising at least one intercalator pseudonucleotide and an essentially complementary DNA (DNA hybrid) is significantly higher than the melting temperature of a duplex consisting of the essentially complementary DNA and a DNA complementary thereto.

Accordingly, oligonucleotides or oligonucleotide analogues may form hybrids with DNA with higher affinity than naturally occurring nucleic acids. The melting temperature is preferably increased with 2 to 30° C., for example from 5 to 20° C., such as from 10° C. to 15° C., for example from 2° C. to 5° C., such as from 5° C. to 10° C., such as from 15° C. to 20° C., for example from 20° C. to 25° C., such as from 25° C. to 30° C., for example from 30° C.; to 35° C., such as from 35° C. to 40° C., for example from 40° C. to 45° C., such as from 45° C. to 50° C. higher.

In particular, the increase in melting temperature may be achieved due to intercalation of the intercalator, because the intercalation may stabilise a DNA duplex. Accordingly, it is preferred that the intercalator is capable of intercalating between nucleobases of DNA. Preferably, the intercalator pseudonucleotides are placed as bulge insertions or end insertions in the duplex (see below), which in some nucleic acids or nucleic acid analogues may allow for intercalation.

The melting temperature of an oligonucleotide or oligonucleotide analogue comprising at least one intercalator pseudonucleotide and an essentially complementary RNA (RNA hybrid) or a RNA-like nucleic acid analogue (RNA-like hybrid) can be significantly higher than the melting temperature of a duplex consisting of the essentially complementary RNA or RNA-like target and the oligonucleotide analogue comprising no intercalator pseudonucleotides. Preferably most or all of the intercalator pseudonucleotides of the oligonucleotide or oligonucleotide analogue are positioned at either or both ends.

Accordingly, oligonucleotides and/or oligonucleotide analogues may form hybrids with RNA or RNA-like nucleic acid analogues or RNA-like oligonucleotide analogues with higher affinity than naturally occurring nucleic acids. The melting temperature is preferably increased with from 2 to 20° C., for example from 5 to 15° C., such as from 10° C. to 15° C., for example from 2° C. to 5° C., such as from 5° C. to 10° C., such as from 15° C. to 20° C. or higher.

The intercalator pseudonucleotides will preferably only stabilise towards RNA and RNA-like targets when positioned at the end of the oligonucleotide or oligonucleotide analogue. This does not however exclude the positioning of intercalator pseudonucleotides in oligonucleotides or oligonucleotide analogues to be hybridized with RNA or RNA-like nucleic acid analogues such that the intercalator pseudonucleotides are placed in regions internal to the formed hybrid. This may be done to obtain certain hybrid instabilities or to affect the overall 2D or 3D structure of both intra- and inter-molecular complexes to be formed subsequent to hybridisation.

An oligonucleotide and/or oligonucleotide analogue comprising one or more intercalator pseudonucleotides may form a triple stranded structure (triplex-structure) consisting of the oligonucleotide and/or oligonucleotide analogue bound by Hoogsteen base pairing to a homologous complementary nucleic acid or nucleic acid analogue or oligonucleotide or oligonucleotide analogue. The oligonucleotide or oligonucleotide analogue may increase the melting temperature of the Hoogsteen base pairing in the triplex-structure.

The oligonucleotide or oligonucleotide analogue may increase the melting temperature of the Hoogsteen base pairing in the triplex-structure in a manner not dependent on the presence of specific sequence restraints like purine-rich/pyrimidine-rich nucleic acid or nucleic acid analogue duplex target sequences. Accordingly, the Hoogsteen base pairing in the triplex-structure has significantly higher melting temperature than the melting temperature of the Hoogsteen base pairing to the duplex target if the oligonucleotide or oligonucleotide analogue had no intercalator pseudonucleotides.

Accordingly, oligonucleotides or oligonucleotide analogues may form triplex-structures with homologous complementary nucleic acid or nucleic acid analogue or oligonucleotide or oligonucleotide analogue with higher affinity than naturally occurring nucleic acids. The melting temperature is preferably increased with from 2 to 50° C., such as from 2 to 40° C., such as from 2 to 30° C., for example from 5 to 20° C., such as from 10° C. to 15° C., for example from 2° C. to 5° C., such as from 5° C. to 10° C., for example 10°C. to 15° C., such as from 15° C. to 20° C., for example from 20° C. to 25° C., such as from 25° C. to 30° C., for example from 30° C. to 35° C., such as from 35° C. to 40° C., for example from 40° C. to 45° C., such as from 45° C. to 50° C.

In particular, the increase in melting temperature may be achieved due to intercalation of the intercalator, because the intercalation may stabilise a DNA triplex. Accordingly, it is preferred that the intercalator is capable of intercalating between nucleobases of a triplex-structure. Preferably, the intercalator pseudonucleotide is placed as a bulge insertion in the duplex (see below), which in some nucleic acids or nucleic acid analogues may allow for intercalation.

Triplex-formation may or may not proceed in strand invasion, a process where the Hoogsteen base-paired third strand invades the target duplex and displaces part or all of the identical strand to form Watson-Crick base pairs with the complementary strand. This can be exploited for several purposes. The oligonucleotides and oligonucleotides are suitably used if only double stranded nucleic acid or nucleic acid analogue target is present and it is not possible, feasible or wanted to separate the target strands, detection by single strand invasion of the region or double strand invasion of complementary regions, without prior melting of double stranded nucleic acid or nucleic acid analogue target, for triplex-formation and/or strand invasion. Accordingly, an oligonucleotide or oligonucleotide analogue comprising at least one intercalator pseudonucleotide is provided that is able to invade a double stranded region of a nucleic acid or nucleic acid analogue molecule.

An oligonucleotide or oligonucleotide analogue comprising at least one intercalator pseudonucleotide that is able to invade a double stranded nucleic acid or nucleic acid analogue in a sequence specific manner can be provided. Invading oligonucleotide and/or oligonucleotide analogue comprising at least-one intercalator pseudonucleotide will bind to the complementary strand in a sequence specific manner with higher affinity than the strand displaced.

The melting temperature of a hybrid consisting of an oligonucleotide analogue comprising at least one intercalator pseudonucleotide and a homologous complementary DNA (DNA hybrid), is usually significantly higher than the melting temperature of a hybrid consisting of the oligonucleotide or oligonucleotide analogue and a homologous complementary RNA (RNA hybrid) or RNA-like nucleic acid analogue target or RNA-like oligonucleotide analogue target. The oligonucleotide may be any of the above described oligonucleotide analogues. For example, the oligonucleotide may be a DNA oligonucleotide (analogue) comprising at least one intercalator pseudonucleotide or a Homo-DNA, b-D-Altropyranosyl-NA, b-D-Glucopyranosyl-NA, b-D-Allopyranusyl-NA, HNA, MNA, ANA, LNA, CNA, CeNA, TNA, (2′-NH)-TNA, (3′-NH)-TNA, □-L-Ribo-LNA, □-L-Xylo-LNA, □-D-Xylo-LNA, □-D-Ribo-LNA, [3.2.1]-LNA, Bicyclo-DNA, 6-Amino-Bicyclo-DNA, 5-epi-Bicyclo-DNA, □-Bicyclo-DNA, □-Bicyclo-DNA, Bicyclo[4.3.0]-DNA, Bicyclo[3.2.1]-DNA, Bicyclo[4.3.0]amide-DNA, □-D-Ribopyranosyl-NA, □-L-Lyxopyranosyl-NA, 2′-R-RNA, 2′-OR-RNA, □-L-RNA, α-D-RNA, β-D-RNA oligonucleotide or mixtures hereof comprising at least one intercalator pseudonucleotide.

Accordingly, the affinity of the oligonucleotide or oligonucleotide analogue for DNA is significantly higher than the affinity of the oligonucleotide or oligonucleotide analogue for RNA or an RNA-like target. Hence in a mixture comprising a limiting number of the oligonucleotide or oligonucleotide analogue and a homologous complementary DNA and a homologous complementary RNA or homologous complementary RNA-like target, the oligonucleotide or oligonucleotide analogue will preferably hybridize to the homologous complementary DNA.

Preferably, the melting temperature of the DNA hybrid is at least 2° C., such as at least 5° C., for example at least 10° C., such as at least 15° C., for example at least 20° C., such as at least 25° C., for example at least 30° C., such as at least 35° C., for example at least 40° C., such as from 2 to 30° C., for example from 5 to 20° C., such as from 10° C. to 15° C., for example from 2° C. to 5° C., such as from 5° C. to 10° C., for example from 10° C. to 15° C., such as from 15° C. to 20° C., for example from 20° C. to 25° C., such as from 25° C. to 30° C., for example from 30° C. to 35° C., such as from 35° C. to 40° C., for example from 40° C. to 45° C., such as from 45° C. to 50° C., for example from 50° C. to 55° C., such as from 55° C. to 60° C. higher than the melting temperature of a homologous complementary RNA or RNA-like hybrid.

An oligonucleotide or oligonucleotide analogue containing at least one 30 intercalator pseudonucleotide can be hybridized to secondary structures of nucleic acids or nucleic acid analogues. The oligonucleotide or oligonucleotide analogue is capable of stabilizing such a hybridization to the secondary structure. Secondary structures could be, but are not limited to, stem-loop structures, Faraday junctions, fold-backs, H-knots, and bulges. The secondary structure can be a stem-loop structure of RNA, where an oligonucleotide or oligonucleotide analogue comprising at least one intercalator pseudonucleotide is designed in a way so the intercalator pseudonucleotide is hybridizing at the end of one of the three duplexes formed in the three-way junction between the secondary structure and the oligonucleotide or oligonucleotide analogue.

Position of Intercalator Pseudonucleotide

An oligonucleotide or oligonucleotide analogue can be designed in a manner so it may hybridize to a homologous complementary nucleic acid or nucleic acid analogue (target nucleic acid). Preferably, the oligonucleotide or oligonucleotide analogue may be substantially complementary to the target nucleic acid. More preferably, at least one intercalator pseudonucleotide is positioned so that when the oligonucleotide analogue is hybridized with the target nucleic acid, the intercalator pseudonucleotide is positioned as a bulge insertion, i.e. the upstream neighbouring nucleotide of the intercalator pseudonucleotide and the downstream neighbouring nucleotide of the intercalator pseudonucleotide are hybridized to neighbouring nucleotides in the target nucleic acid.

An intercalator pseudonucleotide can be positioned next to either or both ends of a duplex formed between the oligonucleotide analogue comprising the intercalator pseudonucleotide and its target nucleotide or nucleotide analogue, for example the intercalator pseudonucleotide may be positioned as a dangling, co-stacking end. All intercalator pseudonucleotides or INA of an oligonucleotide or oligonucleotide analogue can be positioned so that when the oligonucleotide analogue is hybridized with the target nucleic acid, all intercalator pseudonucleotides are positioned as bulge insertions and/or as dangling, co-stacking ends.

Examples of oligonucleotides containing intercalator pseudonucleotides are depicted below:
N₁—(P)_{q}—N₂,
N₁—(P—N₃)_{q}—N₂,
(P)_{q}—N₂,
N₁—(P)_{q},
(P)_{q}—N₂—(P)ᵣ,
N₁—(P)_{q}—N₂,
N₁—(P—N₃)_{q}—N₂—(P—N₃)ᵣN₄,
wherein

N₁, N₂, N₃, N₄ individually denotes a sequence of nucleotides and/or nucleotides analogues of at least one nucleotide,

P denotes an intercalator pseudonucleotide, and

q and r are individually selected from an integer of from 1 to 10.

Methylation

The amount or degree of methylation of genomic DNA has implications in many conditions such as aging, stem cell differentiation, genetic abnormalities, cancer and other disease states. A number of important implications of methylation states were set out below.

The fusion of Embryonic Stem Cells with adult thymocytes to examine the reprogramming that occurs at the level of DNA methylation after the fusion has been made. The inactive somatic X becomes activated as visualized by whole chromosome examination (Tada et al., 2001; Current Biology, 11, 1553-1558).

Examination of methylation patterns in specific DNA regions in the clinicopathological features of sporadic colorectal cancers, as an inexpensive and accurate way of identifying such tumors (Ward et al., 2001; Gut, 48,821-829), and the methylation patterns in stem cells in human colon crypts (Ro et al., 2001, Proc Natl Acad Sci, USA, 98, 10519-10521; Yatabe et al., 2001, Proc. Natl. Acad Sci USA, on line edition).

Methylation patterns in prostate cancer, and in cell lines treated with 5-azacytidine in order to reactivate specific genes (Chetcuti et al., 2001, Cancer Research, 61,6331-6334).

Methylation patterns in the various Estrogen receptors in uterine endometrial cancers where gene inactivation via methylation occurs in many cancers but is not at a high frequency in normal individuals (Sasaki et al., 2001, Cancer Research, 61, 3262-3266).

Methylation patterns in bladder cancer (Markl et al., 2001, Cancer Research, 61, 5875-5884).

Methylation patterns in breast cancer (Nielsen et al., 2001, Cancer Letters, 163, 59-69).

Methylation patterns in specific promoters involved in lung and breast cancers (Burbee et al., 2001, J Natl Cancer Institute, 93, 691-699).

Methylation patterns in free DNA in the plasma of patients with esophageal adenocarcinomas (Kawakami et al., 2000, J Natl Cancer Institute, 92, 1805-1811).

Methylation of the CDHI promoter in hereditary diffuse gastric cancer (Grady et al., 2000, Nature Genetics, 26,16-17).

Genomic imprinting, in which, for example, a paternal allele of a gene is active, and the maternal allele is inactive, or vice versa. This inactivation is accomplished via methylation changes in the genes involved, or in sequences nearby to them. In essence, DNA regions become methylated in the germ line of one sex, but not in that of another (Mann, 2001, Stem Cells, 19,287-294).

Genome-wide methylation patterns in studies of cloning of various species (sheep, cattle, goats, pigs and mice), via nuclear transfer or in vitro fertilization. Thus the methylation patterns of donor nuclei that were inserted into oocytes vary greatly, and this is thought to be the reason why there is such a high failure rate in current cloning experiments. These differentiated nuclei probably require more reprogramming that less differentiated ones such as in Embryonic Stem Cells (Kang et al, 2001; Nature Genetics, 28, 173-177; Humphreys et al., 2001, Science, 293, 95-97).

Excessive hyper-methylation patterns in 24 cancer cell lines versus normal tissues (Smiraglia et al., 2001, Human Molecular Genetics, 10, 1413-1419).

Insertion of methylated DNA into a non methylated mini gene construct to examine the effects on gene expression and imprinting (Holmgren et al., 2001,.Current Biology, 11, 1128-1130).

Methylation patterns in mature B cell lymphomas, where specific genes were inactivated by methylation (Malone et al., 2001, Proc Natl Acad Sci USA 98, 10404-10409).

Methylation patterns of particular genes in acute myeloid leukemia (Melki et al., 1999, Leukemia, 13, 877-883).

Analysis of the Mecp2 gene in knockout mice. This protein is involved in binding o methylated sites in DNA and is thought to be involved in Rett syndrome, which is an inherited neurological disorder (Guy et al., Nature Genetics, 27, 322-326).

Methylation patterns of 5 specific genes during the normal aging process, and in ulcerative colitis (Issa et al., 2001,, Cancer Research, 61, 3573-3577).

Loss of methylation in the processes of apoptosis, which impinge upon signal transduction pathways, cell cycle control, movement of mobile elements within the genome (Jackson-Grusby et al., 2001, Nature Genetics, 27, 31-39).

Comparison of the methylation patterns of promoter and gene regions in different species, such as human and mouse, to determine the evolutionary conservation or lack thereof of CpG islands involved in gene regulation (Cuadrado et al., 2001, EMBO Reports, 21, 586-592).

DNA methylation patterns in testicular sperm at different developmental stages (Manning et al., 2001, Urol Int, 67, 151-155).

Immuno histochemical staining using a monoclonal antibody to analyze DNA methylation patterns (Piyathilake et al., 2000, Biotechnic and Histochem, 75, 251-258).

Differences between the methylation patterns of genes and pseudogenes (Grunau et al., 2000, Human Mol Genet, 9, 2651-2663).

5-methylycytosine content of model invertebrates such as Drosophila melanogaster (Gowher et al., 2000, EMBO J, 19, 6918-6923).

Large scale mapping of human promoters using the methylation patterns of CpG islands (Ioshikhes et al, 2000, Nature Genetics, 26, 61-63).

Induced changes in the processes of chromatin remodelling, DNA methylation and gene expression during mammalian development due to changes in the expression of the ATRX gene which give rise to mental retardation, facial dysmorphism, urogenital abnormalities and alpha thalassemia (Gibbons et al., 2000, Nature Genetics, 24, 368-371).

Boundaries between methylated and unmethylated domains in the promoter region of the GSTP1 gene involved in prostate cancer (Millar et al., 2000, J Biological Chemistry, 275, 24893-24899; Millar et al., 1999, Oncogene, 18, 1313-1324).

Methylation changes during the normal processes of aging (Toyota et al., 1999, Seminars in Cancer Biology, 9, 349-357).

Methylation changes in aging and in atherosclerosis in the cardiovascular system, (Post et al., 1999, Cardiovascular Research, 43, 985-991) and during normal aging and cancers in colorectal mucosa (Ahuja et al., 1998, Cancer Research, 58, 5489-5494).

Methylation patterns in germ cells and sertoli cells in testis (Coffigny et al., 1999, Cytogenet Cell Genets, 87,175-181).

DNA methylation changes during the development of model vertebrates such as the zebrafish (Macleod et al., 1999, Nature Genetics, 23,139-140).

Methylation patterns in the promoter regions of the human histo-blood ABO genes (Kominato et al., 1999, J Biol Chem, 274, 37240-37250).

Methylation patterns during mammalian preimplantation development using monoclonal antibodies (Rougier et al., 1999, Genes and Development, 12, 2108-2113).

Methylation patterns induced by various cancer chemotherapeutic drugs (Nyce, 1997, Mutation Research, 386, 153-161; Nyce 1989, Cancer Research, 49, 5829-5836) and the changes in DNA methylation in phenobarbital-induced and spontaneous liver tumors (Ray et al., 1994, Molecular Carcinogenesis 9, 155-166).

Analysis of 5-methycytosine residues in DNA by the bisulfite sequencing method (Grigg, 1996, DNA Sequence, 6,189-198).

Isolation of CpG islands using a methylated DNA binding column (Cross et al., 1994, Nature Genetics, 6, 236-244).

Is KSHV lytic growth induced by a methylation-sensitive switch? (Laman and Boshoff, Trends Microbiol 2001 October; 9(10):464-6). Both latent and lytic growth of Kaposi's sarcoma-associated herpesvirus (KSHV or HHV-8) contribute to its pathogenesis.

As can be seen from the large number of examples of different methylation states and implications provided above, it will be appreciated that the present invention offers a powerful tool for the study of methylation and thus is useful for many aspects of disease and health.

Materials and Methods

Solid Supports

Table 1 shows some examples of solid supports useful for attaching capture ligands of the present invention. Table 2 shows possible choices of detector systems for use in the present invention.

TABLE 1
Solid supports for attachment of capture ligands

| | | | | | p/ | | |
|---|---|---|---|---|---|---|---|
| | fluoro | | magnetic | latex | styrene | mem- | |
| label | bead | column | bead | bead | bead | brane | glass |
| INA | + | + | + | + | + | + | + |
| Oligo | + | + | + | + | + | + | + |
| RNA | + | + | + | + | + | + | + |
| Chimera | + | + | + | + | + | + | + |

TABLE 2
Detection systems for detection ligands

| | | | | p/ | | |
|---|---|---|---|---|---|---|
| | Fluoro | Magnetic | latex | styrene | | |
| Label | Bead | bead | bead | bead | Glass | Aptamer |
| pre-label | + | | | | | |
| fluorescence | + | + | + | + | + | + |
| Chemi- | + | + | + | + | + | + |
| luminescence | | | | | | |
| radiolabel | + | + | + | + | + | + |
| Dendrimer | + | + | + | + | + | + |

Intercalating Nucleic Acid (INA)

Intercalating nucleic acids (INA) are non-naturally occurring polynucleotides which can hybridize to nucleic acids (DNA and RNA) with sequence specificity. INA are candidates as alternatives/substitutes to nucleic acid probes in probe-based hybridization assays because they exhibit several desirable properties. INA are polymers which hybridize to nucleic acids to form hybrids which are more thermodynamically stable than a corresponding nucleic acid/nucleic acid complex. They are not substrates for the enzymes which are known to degrade peptides or nucleic acids. Therefore, INA should be more stable in biological samples, as well as, have a longer shelf-life than naturally occurring nucleic acid fragments. Unlike nucleic acid hybridization which is very dependent on ionic strength, the hybridization of an INA with a nucleic acid is fairly independent of ionic strength and is favoured at low ionic strength under conditions which strongly disfavour the hybridization of nucleic acid to nucleic acid. The binding strength of INA is dependent on the number of intercalating groups engineered into the molecule as well as the usual interactions from hydrogen bonding between bases stacked in a specific fashion in a double stranded structure. Sequence discrimination is more efficient for INA recognizing DNA than for DNA recognizing DNA.

INA are synthesized by adaptation of standard oligonucleotide synthesis procedures in a format which is commercially available.

There are indeed many differences between INA probes and standard nucleic acid probes. These differences can be conveniently broken down into biological, structural, and physico-chemical differences. As discussed above and below, these biological, structural, and physico-chemical differences may lead to unpredictable results when attempting to use INA probes in applications were nucleic acids have typically been employed. This non-equivalency of differing compositions is often observed in the chemical arts.

With regard to biological differences, nucleic acids are biological materials that play a central role in the life of living species as agents of genetic transmission and expression. Their in vivo properties are fairly well understood. INA, however, is a recently developed totally artificial molecule, conceived in the minds of chemists and made using synthetic organic chemistry. It has no known biological function.

Structurally, INA also differs dramatically from nucleic acids. Although both can employ common nucleobases (A, C, G, T, and U), the composition of these molecules is structurally diverse. The backbones of RNA, DNA and INA are composed of repeating phosphodiester ribose and 2-deoxyribose units. INAs differ from DNA or RNA in having one or more large flat molecules attached via a linker molecule(s) to the polymer. The flat molecules intercalate between bases in the complementary DNA stand opposite the INA in a double stranded structure.

The physico/chemical differences between INA and DNA or RNA are also substantial. INA binds to complementary DNA more rapidly than nucleic acid probes bind to the same target sequence. Unlike DNA or RNA fragments, INAs bind poorly to RNA unless the intercalating groups are located in terminal positions. Because of the strong interactions between the intercalating groups and bases on the complementary DNA strand, the stability of the INA/DNA complex is higher than that of an analogous DNA/DNA or RNA/DNA complex.

Unlike other DNA such as DNA or RNA fragments or PNAs, INAs do not exhibit self aggregation or binding properties.

In summary, as INAs hybridize to nucleic acids with sequence specificity, INAs are useful candidates for developing probe-based assays and are particularly adapted for kits and screening assays. INA probes, however, are not the equivalent of nucleic acid probes. Consequently, any method, kits or compositions which could improve the specificity, sensitivity and reliability of probe-based assays would be useful in the detection, analysis and quantitation of DNA containing samples. INAs have the necessary properties for this purpose.

An example of an INA used for the examples in the present invention was the phosphoramidite of (S)-1-O-(4,4′-dimethoxytriphenylmethyl)-3-O-(1-pyrenylmethyl)-glycerol. It will be appreciated, however, that other chemical forms of INAs can also be used.

Sodium Bisulfite—a Specific Deamination Method

Standard methods for treating nucleic acid with sodium bisufite can be found in a number of references including Frommer et al 1992, Proc Natl Acad Sci 89:1827-1831; Grigg and Clark 1994 BioAssays 16:431436; Shapiro et al 1970, J Amer Chem Soc 92:422 to 423; Wataya and Hayatsu 1972, Biochemistry 11:3583 -3588. Some improvements to these protocols have also been developed by the present inventors.

Detection Systems

Coating Magnetic Beads

The INA, DNA, PNA, LNA, HNA, ANA, MNA, CNA used for attachment to the magnetic beads can be modified in a number of ways. In this example, the INA contained either a 5′ or 3′ amino group for the covalent attachment of the INA to the beads using a hetero-bifunctional linker such as is used EDC. However, the INA can also be modified with 5′ groups such as biotin which can then be passively attached to magnetic beads modified with avidin or steptavidin groups.

Ten μl of carboxylate modified Magnabind™ beads (Pierce) or 100 μl of Dynabeads™ Streptavidin (Dynal) were transferred to a clean 1.5 ml tube and 90 μl of PBS solution added to the magnetic beads.

The beads were mixed then magnetised and the supernatant discarded. The beads were washed ×2 in 100 μl of PBS per wash and finally resuspended in 90 μl of 50 mM MES buffer pH 4.5 or another buffer as determined by the manufactures' specifications.

One μl of 250 μM INA, DNA, PNA, LNA, HNA, ANA, MNA, CNA (concentration dependent on the specific activity of the selected INA as determined by oligonucleotide hybridisation experiments) is added to the sample and the tube vortexed and left at rooin temperature for 10-20 minutes.

Ten μl of a freshly prepared 25 mg/ml EDC solution (Pierce/Sigma) is then added, the sample vortexed and incubated at either room temperature or 4° C. for up to 60 minutes.

The samples were then magnetised, the supernatant discarded and the beads, if necessary, were blocked by the addition of 100 μl either 0.25 M NaOH or 0.5 M Tris pH 8.0 for 10 minutes.

The beads were then washed ×2 with PBS solution and finally resuspended in 100 μl PBS solution.

Hybridisation Using the Magnetic Beads

Ten μl of INA coated Magnabind™ beads were transferred to a clean tube and 40 μl of either ExpressHyb™ buffer (Clontech) either neat or diluted 1:1 in distilled water or any other commercial or in-house hybridization buffer. The buffers may also contain either cationic/anionic or zwittergents at known concentration or other additives such as Heparin and poly amino acids.

Heat denatured sample of DNA 1-5 μl was then added to the above solution and the tubes vortexed and then incubated at 55° C. or another temperature depending on the melting temperature of the chosen INA for 20-60 minutes.

The samples were magnetised and the supernatant discarded and the beads washed ×2 with 0.1×SSC/0.1%SDS at the hybridisation temperature from earlier step for minutes per wash, magnetising the samples between washes.

Dual INA Capture

INA#1 was coupled to a carboxylate modified magnetic bead via a N- or C-terminal amine of the INA and washed to remove unbound INA.

The INA/bead complex was then hybridised to the target DNA in solution using appropriate hybridisation and washing conditions.

The target DNA was then released from the magnetic bead using appropriate methods and transferred to a tube containing a second INA/magnetic beads complex targeted to the opposite end of the DNA molecule.

The second INA/bead complex or oligo/bead complex was then hybridised to the target DNA in solution using appropriate hybridisation and washing conditions.

A third INA or oligonucleotide complementary to the central region of the target DNA could be used as a detector molecule. This detector molecule can be labelled in a number of ways.
(i) The INA, DNA, PNA, LNA, HNA, ANA, MNA, CNA can be directly labelled with a radioactive isotope such as P³² or I¹²⁵ and then hybridised with the target DNA.
(ii) The INA, DNA, PNA, LNA, HNA, ANA, MNA, CNA can be labelled with a fluorescent molecule such as Cy-3 or Cy-5 and then hybridised with the target DNA.
(iii) An amine modified INA, DNA, PNA, LNA, HNA, ANA, MNA, CNA can be labelled in either of the above ways then coupled to a carboxylate modified microsphere of known size then the sphere washed to remove unbound labelled INA, PNA or oligo. This bead complex can then be used to produce a signal amplification system for the detection of the specific DNA molecule.
(iv) The INA, DNA, PNA, LNA, HNA, ANA, MNA, CNA can be attached to a dendrimer molecule either labelled with fluorescent or radioactive groups and this complex used to produce a signal amplification.
(v) The INA, DNA, PNA, LNA, HNA, ANA, MNA, CNA labelled in any of the above ways and hybridised to the target DNA on a solid support can be released into solution using a single stranded specific nuclease such a mung bean nuclease or S1 nuclease. The released detector molecule can be read in a suitable device.
   Preparation of Radio-Labelled Detector Spheres

An INA, DNA, PNA, LNA, HNA, ANA, MNA, CNA can be either 3′ or 5′ labelled with a molecule such as an amine group, thiol group or biotin.

The labelled molecule can also have a second label such as P³² or I¹²⁵ incorporated at the opposite end of the molecule to the first label.

This dual labelled detector molecule can be covalently coupled to a carboxylate or modified latex bead for example of known size using a hetero-bifunctional linker such as EDC. Other suitable substrates can also be used depending on the assay.

The unbound molecules can then be removed by washing leaving a bead coated with large numbers of specific detector/signal amplification molecules.

These beads can then be hybridised with the DNA sample of interest to produce signal amplification.

Preparation of Fluorescent Labelled Detector Spheres

An INA, DNA, PNA, LNA, HNA, ANA, MNA, CNA can be either 3′ or 5′ labelled with a molecule such as an amine group, thiol group or biotin.

The labelled molecule can also have a second label such as Cy-3 or Cy-5 incorporated at the opposite end of the molecule to the first label.

This dual labelled detector molecule can now be covalently coupled to a carboxylate or modified latex bead of known size using a hetero-bifunctional linker such as EDC.

The unbound molecules can then be removed by washing leaving a bead coated with large numbers of specific detector/signal amplification molecules.

These beads can then be hybridised with the DNA sample of interest to produce signal amplification.

Preparation of Enzyme Labelled Detector Spheres

An INA, DNA, PNA, LNA, HNA, ANA, MNA, CNA can be either 3′ or 5′ labelled with a molecule such as an amine group or a thiol group.

The labelled molecule can also have a second label such as biotin or other molecules such as horse-radish peroxidase or alkaline phosphatase conjugated or via a hetero-bifunctional linker at the opposite end of the molecule to the first label.

This dual labelled detector molecule can now be covalently coupled to a carboxylate or modified latex bead of known size using a hetero-bifunctional linker such as EDC.

The unbound molecules can then be removed by washing leaving a bead coated with large numbers of specific detector/signal amplification molecules.

These beads can then be hybridised with the DNA sample of interest to produce signal amplification.

Signal amplification can then be achieved by binding of a molecule such as streptavidin or an enzymatic reaction involving a calorimetric substrate.

INA Oligomer Combinations

In all of the above cases the initial hybridization event involved the use of magnetic beads coated with an INA complementary to the nucleic acid of interest.

The second hybridisation event can involve any of the methods mentioned above.

This hybridisation reaction can be done with either a second INA complementary to the DNA of interest, a PNA or an oligonucleotide or modified oligonucleotide complementary to the nucleic acid of interest. As fluorescent beads of convenient size in these assays, carry >10⁶fluorochrome molecules and a single fluorescent bead can be detected readily, the method has the potential sensitivity to assay one or a few DNA molecules from one or a few cells.

Dendrimers and Aptamers

Dendrimers are branched tree-like molecules that can be chemically synthesised in a controlled manner so that multiple layers can be generated that were labelled with specific molecules. They were synthesised stepwise from the centre to the periphery or visa-versa.

One of the most important parameters governing dendrimer structure and its generation is the number of branches generated at each step; this determines the number of repetitive steps required to build the desired molecule.

Dendrimers can be synthesised that contain radioactive labels such as I¹²⁵ or P³² or fluorescent labels such as Cy-3 or Cy-5 to enhance signal amplification.

Alternatively dendrimers can be synthesised to contain carboxylate groups or any other reactive group that could be used to attach a modified INA, PNA or DNA molecule.

Methods

Detection of Methylated DNA Using Solid Supports and Magnetic Beads

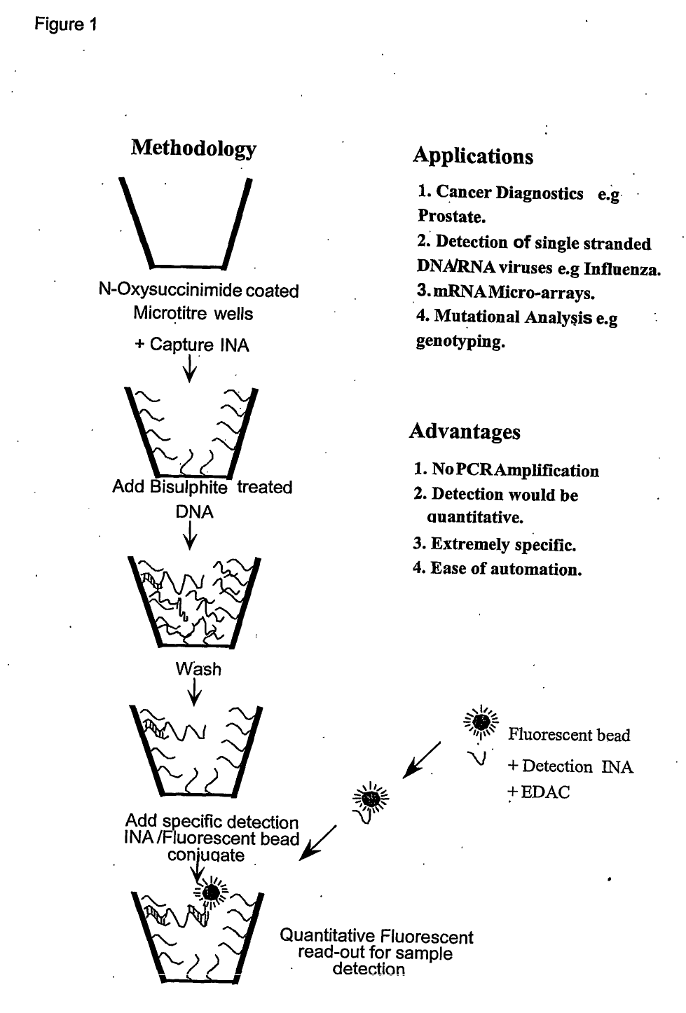

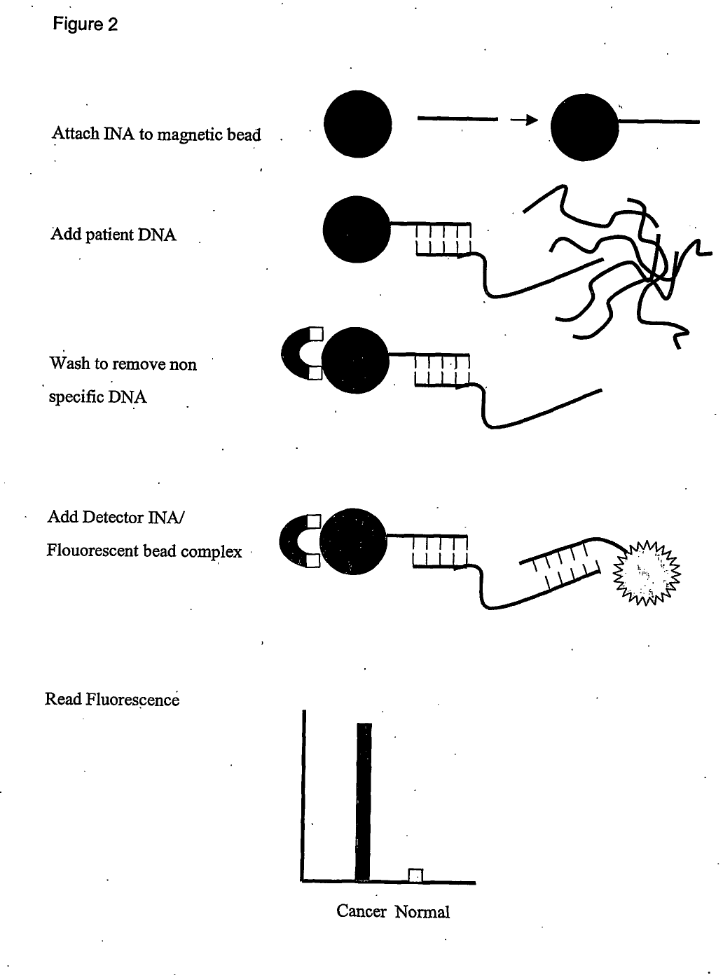

FIG. 1 and FIG. 2 show examples of the method of the invention using sandwich INA signal amplification using solid supports and magnetic beads, respectively. Although INA is exemplified as the detector ligand in FIG. 1 and FIG. 2, it will be appreciated that other detector ligands such as oligonucleotides can be used in these methods.

A solid support in the form of a microtiter well was provided and coated with N-oxysuccinimide to assist in the adhesion of INA or other ligand to the well.

A first INA which was complementary to a first part of the target nucleotide sequence is added to the well and attached to this solid support.

Bisulfite treated DNA was then added to the well and allowed to hybridise with the INA to capture the target DNA which had hybridised to the INA and subsequently bound to the well.

The well was then washed to remove the hybridisation solution and any non-hybridised DNA leaving only the hybridised DNA captured on the well.

Next, a second INA which was complementary to a second part of the target nucleotide sequence was linked to microsphere beads having fluorescent labelling. The second linked INA was then hybridised with the target DNA already bound to the well. The well was then washed to remove the unhybridized second INA/microsphere complex leaving only the INA/microsphere complex and fluorescent label associated with the target DNA sequence.

The fluorescence was then measured to determine the level of target DNA.

Detection of Methylated DNA Using Microspheres

Methodology

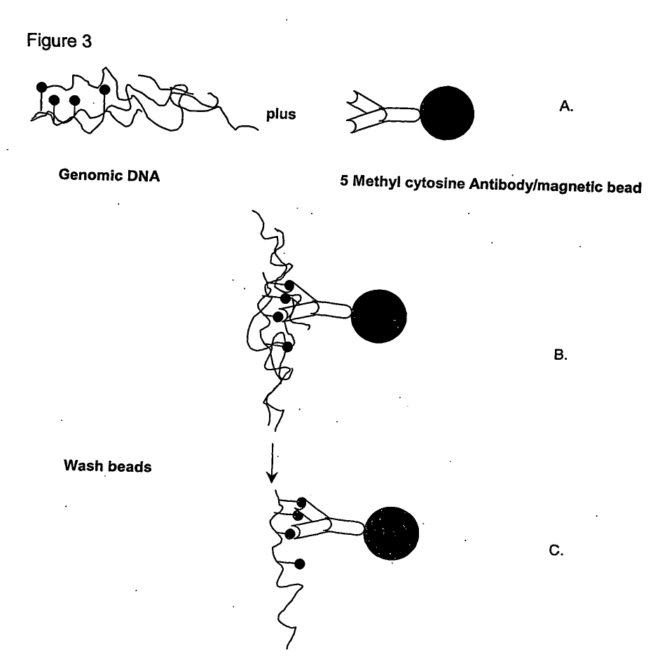

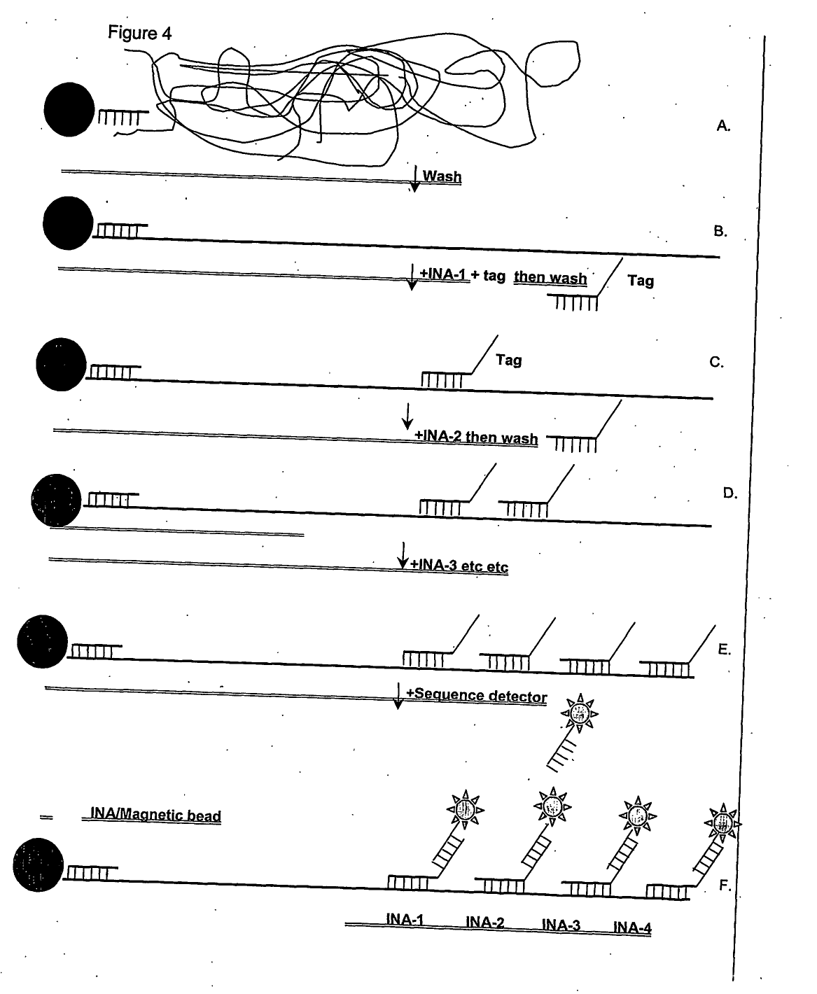

Referring to FIG. 3 and FIG. 4, the detection of methylated DNA using microspheres is shown.

Coating Microtitre Wells with Capture INA

(i) The capture INA (0.01-100 pM per well) in 50 mM Phosphate buffer, 1 mM EDTA pH 8.5 (100 μl) was used to coat N-oxysuccinimide-coated microtitre wells (Costar Cat#2498) for 16-24 hours@4° C.
(ii) Plates were washed with 100 μl of 50 mM Phosphate buffer, 1 mM EDTA pH 8.5.
(iii) 150 μl of 3% BSA, 50 mM Phosphate buffer, 1 mM EDTA pH 8.5 was added to each well and the plates left@4° C. until required.
   Coating the Fluorospheres with Detection INA
(i) Fluorospheres (Molecular Probes) were sonicated five times for 5 seconds to break up any aggregated material.
(ii) The detection probe INA was diluted in a range from 300 pM to 0.3 pM in 250 μl of sonicated 50 mM 2[N-morpholino] ethanesulphonic acid (MES) pH 6.0 and 250 μl of sonicated fluorospheres added and the solution left at room temperature for 30 minutes.
(iii) 0.5 mg of 1-ethyl-3[3 dimethylamine propyl] carbodiimide [EDAC], Sigma Cat #E1769, was added to the sample and the sample left 4-6 hours at room temperature in the dark then incubated 16 hours at 4° C.
(iv) 55 μl of 1 M glycine was added to the beads and the beads left at room temperature for 2 hours.
(v) The beads were centrifuged for 5-20 minutes (dependent on size of beads, generally 0.5 μM beads required 5 mins while 0.1 μM beads required 20 minutes) at 14,000 rpm in a bench top centrifuge and the supernatant discarded.
(vi) Beads were washed twice with 500 μl of PBS/1% BSA with centrifugation as before between wash steps.
(vii) The beads were then resuspended in 200 μl of PBS/1% BSA and stored at 4° C. in the dark until required.
(viii) Variation of the number of INA ligands bound to the beads can be used to optimise sensitivity and minimise background levels.
   Hybridisation of DNA
(i) Either control salmon sperm DNA or DNA that was bisulfite treated as in Clark et al (Clark S J, Harrison J, Paul C L and Frommer M. High sensitivity mapping of methylated cytosines. Nucleic Acids Res. 22: 2990-2997 (1994)) was hybridised with INA ligands coupled to microtitre wells then added to each well.
(ii) DNA samples were mixed with 100 μl of ExpressHyb™ buffer (Clontech), added to the wells and the plate covered with cling film or the wells overlayed with mineral oil (Sigma) for longer incubations and the samples incubated at between 45-60° C. for between 1-16 hours.
(iii) Wells were then washed twice with 150 μl of 2×SSC/O.1%SDS@45-60° C. for 5-10 minutes per wash.
(iv) The wells were further washed with 150 μl of 0.1×SSC/0.1%SDS@45-60° C. for 5-10 minutes and the wash solution discarded.
(v) The INA fluorospheres were diluted 1/100 in ExpressHyb™ buffer (Clontech) and 100 μl of samples added to the wells. The plates were covered with cling film or the wells overlayed with mineral oil (Sigma) for longer incubations and the samples incubated@between 45-60° C. for between 1-16 hours.
(vi) Wells where then washed twice with 150 μl of 2×SSC/0.1%SDS at 45-60° C. for 5-10 minutes per wash.
(vii) The wells were further washed with 150 μl of 0.1×SSC/0.1%SDS at 45-60° C. for 5-10 minutes and the wash solution discarded.
(viii) Finally the fluorescent intensity of each well was measured at the appropriate excitation/emission wave-length for the particular bead (500/520 for yellow beads) in a Victor II fluorescent plate reader.
(ix) Background values measured in wells to which no INA had been attached were subtracted from all readings.
   Method for Production of Coated Radiolabelled Beads
(i) A specific oligonucleotide (INA or PNA) was synthesised against the target DNA or nucleic acid region of interest. This oligonucleotide, INA or PNA contained a 3′ amine group synthesised using standard chemistry (Sigma Genosys).

(ii) The oligonucleotide (INA or PNA) was then 5′ kinased using gamma P³²dATP as follows:

| | |
|---|---|
| Oligonucleotide (20 ng/μl) | 1 μl |
| x10 PNK buffer | 1 μl |
| T4 PNK | 1 μl |
| Gamma P³²dATP | 2 μl |
| Sterile water | 5 μl |

(iii) The sample was then incubated at 37° C. for 1 hour then heated to 95° C. for 5 minutes to inactivate the enzyme.

(iv) 0.1 μM carboxylate modified fluorescent beads (Molecular Probes Cat# F-8803) are diluted 1/10,000, 1/100,000 and 1/1,000,000 in sterile water then the kinased oligonucleotide coupled to the beads as follows:

| | |
|---|---|
| Beads | 1 μl |
| Labelled oligo (INA or PNA) | 3 μl |
| 50 mM MES pH 8.0 | 5 μl |
| 10 mg/ml EDC (Pierce) | 2 μl |

(v) The beads were then incubated@room temperature for 1 hour to allow the kinased oligonucleotide to attach to the beads via the 3′ amine.
(vi) The beads were then spun in a microfuge at full speed for 15 minutes to sediment the coated beads.
(vii) The supernatant was removed and the beads washed with 100 μl of PBS solution and spun as above.
(viii) The supernatant was removed and the beads resuspended in 50 μl of PBS.
(ix) The CPM of the coated beads was then measured in a standard scintillation counter using the Cerenkov counting protocol. The beads with the highest activity were then used as a detector system in the assay

The idea behind this protocol was to produce the smallest number of beads with the highest specific activity, so that only a few beads were needed to bind to the target sequence in order to generate a detectable signal.

Bisulphite Treatment of DNA

To 2 μg of DNA, 2 μl (1/10 volume) of 3 M NaOH (6 g in 50 ml water, freshly made) was added in a final volume of 20 μl. The mixture was incubated at 37° C. for 15 minutes. Incubation at temperatures above room temperature can be used to improve the efficiency of denaturation.

After the incubation, 208 μl 2 M sodium metabisulphite (7.6 g in 20 ml water or Tris/EDTA with 416 ml 10 N NaOH; BDH AnalaR #10356.4D; freshly made) was added. The sample was overlaid with 200 μl of mineral oil. The sample was then incubated overnight at 55° C. Alternatively the samples can be cycled in a thermal cycler as follows: incubate for about 4 hours or overnight as follows: Step 1, 55° C./2 hr cycled in PCR machine; Step 2, 95° C./2 min. Step 1 can be performed at any suitable temperature from about 37° C. to about 90° C. and can vary in length from 5 minutes to 16 hours. Step 2 can be performed at any temperature from about 70° C. to about 99° C. and can vary in length from about 1 second to 60 minutes, or longer.

After the treatment with sodium metabisulphite, the oil was removed, and 1 μl tRNA (20 mg/ml) or 2 μl glycogen were added if the DNA concentration was low. These additives are optional and can be used to improve the yield of DNA obtained by co-precitpitating with the target DNA especially when the DNA is present at low concentrations.

An isopropanol cleanup treatment was performed as follows: 800 μl of water were added to the sample, mixed and then 1 ml isopropanol was added. The sample was mixed again and left at −20° C. for a minimum of 5 minutes. The sample was spun in a microfuge for 10-15 minutes and the pellet was washed 2× with 80% ETOH, vortexing each time. This washing treatment removes any residual salts that precipitated with the nucleic acids.

The pellet was allowed to dry and then resuspended in a suitable volume of T/E (10 mM Tris/0.1 mM EDTA) pH 7.0-12.5 such as 50 μl. Buffer at pH 10.5 has been found to be particularly effective. The sample was incubated at 37° C. to 95° C. for 1 min to 96 hr, as needed to suspend the nucleic acids.

Antibody Approach

Antibody Selection for Methylated DNA Sequences in a Genome

The approach is set out in FIG. 3 and summarized below.
I. An antibody directed against 5-methylcytosine is coated onto magnetic beads. (A)
II. After washing to remove unbound antibody, the beads are added to genomic DNA. (B)
III. Any DNA containing 5-methylcytosine binds to the antibody coated bead leaving the bulk of the unmethylated DNA free in solution.
IV. The antibody/beads are washed to yield a pure population of methylated DNA sequences which can then be subjected to bisulphite treatment. (C)
   Multiple Ligand Approach
   Multiple Ligand Using Intercalating Nucleic Acid ligands (INA)

One preferred approach is set out in FIG. 4. Using this method the sequence of interest is detected as follows:
I. An INA, designed to 5′ region of the sequence of interest, is coupled to a magnetic bead or detectable particle. (A)
II. The INA/bead complex is mixed with bisulphite treated DNA and washed to remove non-target DNA. (B)
III. A second INA, PNA or oligo is then added (it being designed to a 3′ region of the sequence of interest). The second INA, PNA or oligo contains a unique sequence tag not found in the genome which is subsequently used for detection (c)
IV. The sample is then washed and a second INA species is added again containing the same tag and the beads washed. (D)
V. A third and fourth INA etc are added, hybridised and washed. (E)
VI. The tag sequence is now detected using a labelled (Fluorescent/radioactive) INA, PNA or oligo that binds to the tag sequence of the INAs 1-4. (F)

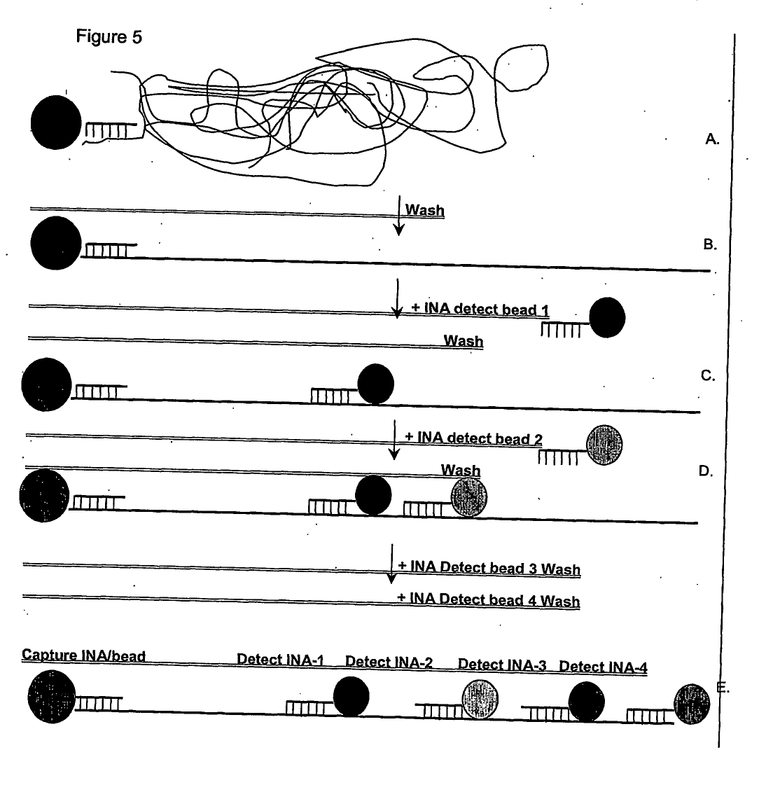

Another preferred approach is set out in FIG. 5. Using this method the sequence of interest is detected as follows:
I. An INA is coupled to a magnetic bead or detectable particle designed to the 5′ region of the sequence of interest. (A, B)
II. The INA/bead complex is mixed with bisulphite treated DNA and washed to remove non-target DNA. (C)
III. A second INA/bead is then added designed to a 3′ region of the sequence of interest. The second INA/bead complex is labelled either fluorescently or radioactively to be used for detection. (D)
IV. The sample is then washed and a third INA/bead complex is added again fluorescently or radioactively labelled. (D)
V. A third and fourth INA etc are added hybridised and washed.
VI. Signal amplification is then achieved using the sum of all the detector bead complexes. (E)
   Antibody Capture Multiple Ligand Assay

The present inventors have found that antibodies directed to 5-methylcytosine (normally used for staining chromosomes) can be used to capture or concentrate nucleic acids having areas of high methylation. Once captured, the nucleic acid can be assayed in accordance with methods according to the present invention.

The assay is described below.

A. Coupling 5-Methylcytosine Antibody to Magnetic Beads

I. 0.1 μl (0.5 μg) of Monoclonal 5-methylcytosine antibody (Oncogene Cat#NA81) was added to 125 μl of Dynal Pan Mouse IgG (cat#110.22), washed according to manufacturers instructions.
II. The samples were rocked at room temperature for 45 minutes.
III. Beads were washed ×4 with PBS/0.1% BSA.
IV. Beads were then resuspended in 125 μl of PBS/0.1% BSA.
   B. Pre-Enrichment of Genomic DNA
I. 6.5 μg of Genomic LNCaP DNA, pre-digested with EcoR1 and HindIII according to the manufacturers instructions, was added to the washed beads.
II. Samples were rocked at room temperature for 45 minutes.
III. Beads were washed ×4 with PBS/0.1%BSA.
IV. Beads were then resuspended in 40 μl of water.
   C. Bisulphite Treatment of Captured DNA
I. 20 μl of captured DNA was bisulphite treated as follows.
II. 2 μl (1/10 volume) of 3 M NaOH. The mixture was incubated at 37° C. for 15 minutes.
III. After the incubation, 208 μl 2 M sodium metabisulphite was added. The sample was overlaid with 200 μl of mineral oil.
IV. The sample was then incubated overnight at 55° C.
V. After the treatment with sodium metabisulphite, the oil was removed, and 1 μl tRNA (20 mg/ml).
VI. 800 μl of water was added to the sample, mixed and then 1 ml isopropanol was added. The sample was mixed again and left at 4° C. for a 30 minutes.
VII. The sample was spun in a microfuge for 10-15 minutes and the pellet was washed 2× with 80% ETOH, vortexing each time.
VIII. The pellet was allowed to dry and then resuspended in 50 μl T/E (10 mM Tris/0.1 mM EDTA) pH 10.5.
IX. The sample was incubated at 72° C. for 1 hr.
   D. Preparation of PNA Capture Beads

The following PNA was synthesised to recognise a methylated sequence of the GSTP1 Gene (Accession number: M24485).

PNA 5′amine-CTA ACG CGC CGA AAC
I. Ten μl of carboxylate modified Magnabind™ beads. (Pierce cat#21353) were transferred to a clean 1.5 ml tube and 90 μl of PBS solution added to the magnetic beads.
II. The beads were mixed then magnetised and the supernatant discarded. The beads were washed ×2 in 100 μl of PBS per wash and finally resuspended in 90 μl of 50 mM MES buffer pH 4.5
Ill. One μl of 250 μM PNA was added to the sample and the tube vortexed and left at room temperature for 10-20 minutes.
IV. Ten μl of a freshly prepared 25 mg/ml EDC solution (Pierce/Sigma) is then added, the sample vortexed and incubated at either room temperature or 4° C. for up to 60 minutes.
V. The samples were then magnetised, the supernatant discarded.
VI. The beads were blocked by the addition of 100 μl either 0.25 M NaOH or 0.5 M Tris pH 8.0 for 10 minutes.
VII. The beads were then washed ×2 with PBS solution and finally resuspended in 100 μl PBS solution.
   E. Hybridisation of the PNA Coated Capture Beads to the Antibody Enriched Bisulphite Treated DNA
I. 10 μl of PNA-coated beads were added to a fresh 1.5 ml centrifuge tube along with 5 μl of antibody enriched bisulphite treated DNA and 35 μl of ExpressHyb solution (Clontech) diluted 1:1 with distilled water.
II. Samples were mixed and left at 55° C. for 1 hour.
III. Samples were washed ×1 with ×2 SSC/0.1% SDS at 55° C. Magnetised and the supernatant discarded.
IV. Samples were washed a further ×1 with ×1SSC/0.1% SDS at 55° C. Magnetised and the supernatant discarded.
V. Finally the samples were resuspended in 20 μl ×1 SSC/0.1% SDS.
   F. Kinasing the Detect Oligonucleotides

Four specific detect oligonucleotides were designed to a methylated region downstream of the region used in the initial PNA capture. The sequence of these primers is shown below:

(SEQ ID NO: 1)

| | |
|---|---|
| DETECT-1 | 5′-TAAATCACGACGCCGACCGCTCTT-amine 3′ |

(SEQ ID NO: 2)

| | |
|---|---|
| DETECT-2 | 5′-AAAACGCGAACCGCGCGTACTCA-amine 3′ |

(SEQ ID NO: 3)

| | |
|---|---|
| DETECT-3 | 5′-CCTAAAAACCGCTMCGACACTA-amine 3′ |

(SEQ ID NO: 4)

| | |
|---|---|
| DETECT-4 | 5′-TAAACCACGATATAAAACGACACTC-amine 3′ |

The synthetic oligonucleotides were kinased as follows:

| | | |
|---|---|---|
| Oligo | 40 | ng |
| x10 buffer | 2 | μl |
| T4 Kinase | 2 | μl |
| Gamma P32 | 4 | μl |
| Water | to 20 | μl |

The reaction was heated at 37° C. for 60 minutes then the enzyme heat denatured at 95° C. for 5 minutes.

G. Attaching the Kinased Oligonucleotides to Fluorescent Beads

I. 5 μl of kinased detect oligos were coupled to 1 μl of a 10⁻⁷ dilution Molecular Probes carboxylate fluorospheres 0.5pM (Cat#F-8812-pink) as follows.

| | |
|---|---|
| 10⁻⁷ fluorospheres 0.5 μM | 1 μl |
| Kinased detect oligo | 5 μl |
| 50 mM MES pH 8.0 | 12 μl |
| 10 mg/ml EDC (Sigma) | 2 μl |

II. The beads were left 1 hour at room temperature.
III. Beads were washed ×1 with SSC/0.1% SDS and resuspended as follows
IV. Detect 1. 20 μl of ×0.1SSC/0.1% SDS
V. Detect 2. 10 μl of ×0.1SSC/0.1% SDS
VI. Detect 3. 10 μl of ×0.1SSC/0.1% SDS
VII. Detect 4. 10 μl of ×0.1SSC/0.1% SDS
   H. Hybridisation of the Detect Oligos to the PNA Captured Antibody Enriched Bisulphite Treated DNA
I. Beads from section F were magnetised and the supernatant removed.
II. 47 μl of ExpressHyb solution (Clontech) diluted 1:1 with distilled water was added to the sample.
III. 3 μl of each of the Detect beads 14 (12 μl total volume) was added to the samples.
IV. Samples were incubated at 55° C. for 1 hour.
V. Samples were washed ×1 with ×2 SSC/0.1% SDS at 55° C. Magnetised and the supernatant discarded.
VI. Samples were washed a further ×1 with ×1SSC/0.1% SDS at 55° C. Magnetised and the supernatant discarded.
VII. Finally the beads were resuspended in 5 ml of InstaGel scintillant and the radioactivity determined by scintillation counting using the Cerenkov protocol.
   Results

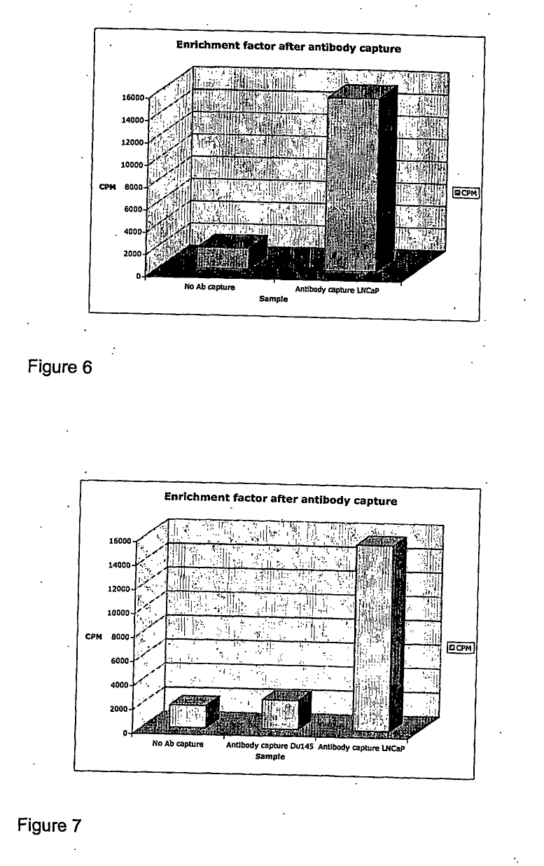

FIG. 6 shows enrichment factor provided when comparing genomic DNA samples that did not receive antibody versus antibody capture samples.

FIG. 7 shows non-PCR signal amplification using the antibody capture multiple ligand assay. The results show signals obtained using 1. no antibody enrichment with LNCaP DNA (methylated DNA), 2. Antibody enriched Du145 DNA (unmethylated DNA) and 3. Antibody enriched LNCaP DNA (methylated DNA).

INA Probes to Capture Genomic DNA Sequences and detection Using PCR

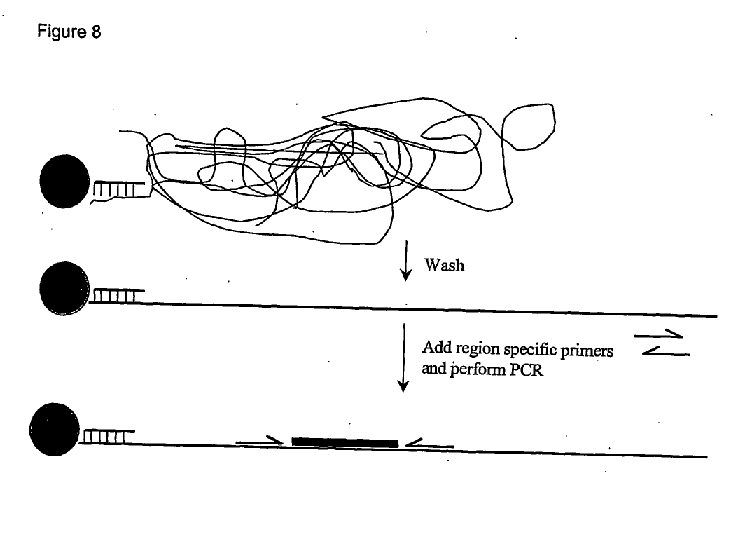

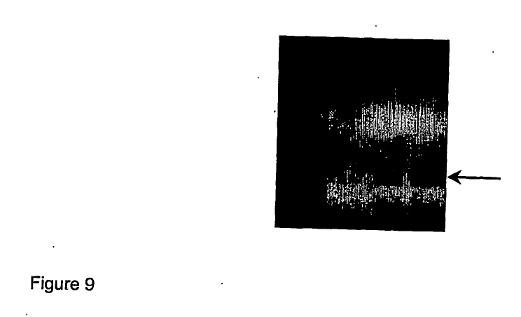

The assay is summarized in FIG. 8. Using this method the sequence of interest is detected as follows:
I. An INA directed to a bisulphite converted methylated region, or a bisulphite converted unmethylated region, are designed to the 5′ region of the sequence of interest and then coupled to a magnetic bead or any solid phase.
II. The INA/bead complex is mixed with bisulphite treated DNA and washed to remove non-target DNA.
III. The captured material is then used as input material for a PCR to detect sequence downstream from the capture site, where a positive PCR indicates the desired sequence was captured.
IV. The INA ligand may also be bound to any particle that is discernible by shape, and therefore many thousands of reactions can occur in a singular reaction tube.
V. INA, PNA or oligos, or the like, are attached to such particles such that INA1 is attached to particle of shape 1 . . . INA2 is attached to particle of shape 2 . . . INA3 to particle of shape 3 . . . etc. the particles are then all put into one tube etc . . . for subsequent reactions.
VI. The INAs may also be physically bound to wells of a PCR plate, and the whole reaction performed in a single well. This allows for a ‘kit’ format where the positive signals generated can be decoded (for methylation/no methylation) by position in the plate (see FIG. 9 below for agarose gel experimental results).
   Coupling Amine Modified Nucleic Acids to Magnetic Beads
1. Ten μl of carboxylate modified Magnabind T beads (Pierce) were transferred to a clean 1.5 ml tube and 90 μl of PBS solution added to the magnetic beads.
II. The beads were mixed then magnetised and the supernatant discarded. The beads were washed ×2 in 100 μl of PBS per wash and finally resuspended in 90 μl of 50 mM MES buffer pH 4.5 or another buffer as determined by the manufactures' specifications.
III. One μl of 250 μM INA, (concentration dependent on the specific activity of the selected INA as determined by oligonucleotide hybridisation experiments) was added to the sample and the tube vortexed and left at room temperature for 10-20 minutes.
IV. Ten μl of a freshly prepared 25 mg/ml EDC solution (Pierce/Sigma) was then added, the sample vortexed and incubated at either room temperature or 4° C. for up to 60 minutes.
V. The samples were then magnetised, the supernatant discarded and the beads, if necessary were blocked by the addition of 100 μl either 0.25 M NaOH or 0.5 M Tris pH 8.0 for 10 minutes.
VI. The beads were then washed ×2 with PBS solution and finally resuspended in 100 μl PBS solution.
   Hybridisation Using the INA Coated Magnetic Beads to Genomic DNA

Ten μl of INA coated Magnabind™ beads were transferred to a clean tube and 40 μl of either ExpressHyb™ buffer (Clontech) either neat or diluted 1:1 in distilled water or any other commercial or in-house hybridization buffer. The buffers may also contain either cationic/anionic or zwittergents at known concentration or other additives such as Heparin and poly amino acids.

Heat denatured sample of DNA 1-5 μl was then added to the above solution and the tubes vortexed and then incubated at 55° C. or another temperature depending on the melting temperature of the chosen INA for 20-60 minutes.

The samples were magnetised and the supernatant discarded and the beads washed ×2 with 0.1×SSC/0.1%SDS at the hybridisation temperature from earlier step for 5 minutes per wash, magnetising the samples between washes.

PCR Amplification of INA Captured DNA

PCR amplification was performed on 1 μl of treated DNA, ⅕^{th} volume of final resuspended sample volume, as follows. PCR amplifications were performed in 25 μl reaction mixtures containing 1 μl of bisulphite treated genomic DNA, using the Promega PCR master mix, 6 ng/μl of each of the primers. The strand-specific nested primers used for amplification of GSTP1 from bisulphite-treated DNA are GST-9 (967-993) TTTGTTGTTTGTTTATTTTTAGGTTT (F) GST-10 (1307-1332) (SEQ ID NO: 5) AACCTAATACTACCAATTAACCCCAT 1^{st} round amplification conditions (SEQ ID NO: 6).

One μl of 1^{st} round amplification was transferred to the second round amplification reaction mixtures containing primers( R) GST-11 (999-1027) GGGATTTGGGAAAGAGGGAAAGGTTTTTT (F) GST-12 (1281-1306) (SEQ ID NO: 7) ACTAAAAACTCTAAAAACCCCATCCC (R) (SEQ ID NO: 8). The location of the primers is indicted according to the GSTP1 sequence (Accession number: M24485). Samples of PCR products were amplified in a ThermoHybaid PX2 thermal cycler under standard conditions.

Agarose gels (2%) were prepared in 1% TAE containing 1 drop ethidium bromide (CLP #5450) per 50 ml of agarose. Five ul of the PCR derived product was mixed with 1 μl of 5× agarose loading buffer and electrophoresed at 125 mA in ×1 TAE using a submarine horizontal electrophoresis tank. Markers were the low 100-1000 bp type. Gels were visualised under UV irradiation using the Kodak UVidoc EDAS 290 system.

FIG. 9 shows agarose gel representation of the INA capture and PCR method. INA ligands specific for an unmethylated genomic DNA sequence were coupled to magnetic beads and were mixed with genomic bisulphite treated DNA. The bead/DNA complex was washed and the bound molecules used as a template in PCR for a downstream region. LANES; MARKER, 1, 2, 3, where,
LANE 1: HepG2 DNA (Known to be methylated at target site),
LANE 2: Du145 DNA (Known to be unmethylated at target site),
LANE 3: BL1 3 DNA (Known to be unmethylated at target site).

The results show that using an INA ligand directed to an unmethylated target nucleic acid coupled to a magnetic bead, the INA is able to specifically capture unmethylated bisulphite treated total genomic DNA. The genomic DNA used in lane 1 (HepG2) is methylated at the genomic loci at which the INA was directed. The genomic DNA used in lane 2 (Du145) and 3 (BL13) is unmethylated at the genomic loci at which the INA was directed resulting in positive PCR signals in both lanes. Furthermore, this example shows that the approach may be used with PCR detection for rapid determination of the presence of methylated/unmethylated target nucleic acids.

Specificity of the INA Ligands Using Methylated Mixtures

Preparation of INA Capture Beads.

The following INA was synthesised to recognise a methylated sequence of the GSTP1 Gene and an unmethylated version of the same region (Accession number: M24485). (Y indicates a pseudo intercalating nucleotide)

Methylated INA-1

(SEQ ID NO: 9)

5′ amine-YA TCY GGC YGC GCY AAC YTA Y
Unmethylated INA-2

(SEQ ID NO: 10)

5′ amine-CTA ACG CGC CGA AAC

I. Ten μl of carboxylate modified Magnabind™ beads (Pierce cat#21353) were transferred to a clean 1.5 ml tube and 90 μl of PBS solution added to the magnetic beads.
II. The beads were mixed then magnetised and the supernatant discarded. The beads were washed ×2 in 100 μl of PBS per wash and finally resuspended in 90 μl of 50 mM MES buffer pH 4.5
III. One μl of 250 μM PNA was added to the sample and the tube vortexed and left at room temperature for 10-20 minutes.
IV. Ten μl of a freshly prepared 25 mg/ml EDC solution (Pierce/Sigma) is then added, the sample vortexed and incubated at either room temperature or 4° C. for up to 60 minutes.
V. The samples were then magnetised, the supernatant discarded.
VI. The beads were blocked by the addition of 100 μl either 0.25 M NaOH or 0.5 M Tris pH 8.0 for 10 minutes.
   The beads were then washed ×2 with PBS solution and finally resuspended in 100 μl PBS solution.
   Hybridisation of the INA Ligands to a Synthetic Methylates and Unmethylated GSTP1 Sequences

Two synthetic 110 bp oligonucleotides were designed to represent a methylated and unmethylated region of the GSTP1 gene.

Unmethytated Sequence

(SEQ ID NO: 11)

5′AGGGAATTTTTTTTTTGTGATGTTTTGGTGTGTTAGTTTGTTGTGTAT
ATTTTGTTGTGGGTTTTTTTTTTGGTTTTTTTGGTTAGTTGTGTGGTGAT
TTTGGGGATTTTAG-3′
Methylated Sequence

(SEQ ID NO: 12)

5′AGGGAATTTTTTTTCGCGATGTTTCGGCGCGTTAGTTCGTTGCGTATA
TTTCGTTGCGGTTTTTTTTTTGGTTTTTTCGGTTAGTTGCGCGGCGATTT
CGGGGATTTTAG-3′

The methylated and unmethylated sequences were then mixed in the following ratios of methylated:unmethylated

```
100:0%
99:1%
95:5%
  90:10%
  75:25%
  50:50%
  25:75%
  10:90%
  5:95%
  1:99%
    0:100%
```

Hybridisating Reaction
I. 40 μl of ExpressHyb solution (Clontech) diluted 1:1 with distilled water was added to the 5 μl of coupled beads.
II. 5 μl oligo mix was added and the solution mixed by vigorous pipetting to resuspend the particles.
III. The samples were then incubated at 50° C. for 30 minutes to allow binding of target sequences.
IV. The beads were magnetised and the supernatant removed.
V. The beads were then washed ×1 with ×2 SSC/0.1% SDS at 50° C. for 5 minutes.
VI. The beads were magnetised and the supernatant removed.
VII. The beads were washed a further ×1 with ×1SSC/0.1% SDS at 50° C. for 5 minutes.
   Kinasing the Detector Oligonucleotides

Two detector oligonucleotides (oligos) were synthesised that bound to a 3′ region of either the methylated or unmethylated synthetic oligo sequence.

Methylated Detector

(SEQ ID NO: 13)

5′-AAA CTA ACA CAC CAA AAC ATC ACA AA-amine-3′
Unmethylated Detector

(SEQ ID NO: 14)

5′-GAA CTA ACG CGC CGA AAC ATC GCG AA-amine-3′

The oligo was kinased as follows

| | | |
|---|---|---|
| Oligo | 100 | ng |
| x10 buffer | 2 | μl |
| T4 Kinase | 2 | μl |
| Gamma P32 | 4 | μl |
| Water | to 20 | μl |

The reaction was heated at 37° C. for 60 minutes then the enzyme heat denatured at 95° C. for 5 minutes. The reaction volume was then adjusted to 55 μl with PCR grade water.
Hybridisation of Kinased Oligonucleotides to INA Capture Magnetic Beads
I. The washed INA capture beads were resuspended in 45 μl of ExpressHyb solution (Clontech) diluted 1:1 with distilled water
II. 5 μl kinased oligo was added and the solution mixed by vigorous pipetting to resuspend the particles.
III. The samples were then incubated at 50° C. for 30 minutes to allow binding of target sequences.
IV. The beads were magnetised and the supernatant removed.
V. The beads were then washed ×1 with ×2 SSC/0.1% SDS at 50° C. for 5 minutes.
VI. The beads were magnetised and the supernatant removed.
VII. The beads were washed a further ×1 with ×1 SSC/0.1% SDS at 50° C. for 5 minutes.
VIII. The beads were magnetised and the supernatant removed.
IX. Finally the beads were resuspended in 5 ml of InstaGel scintillant and the radioactivity determined by scintillation counting using the Cerenkov protocol.

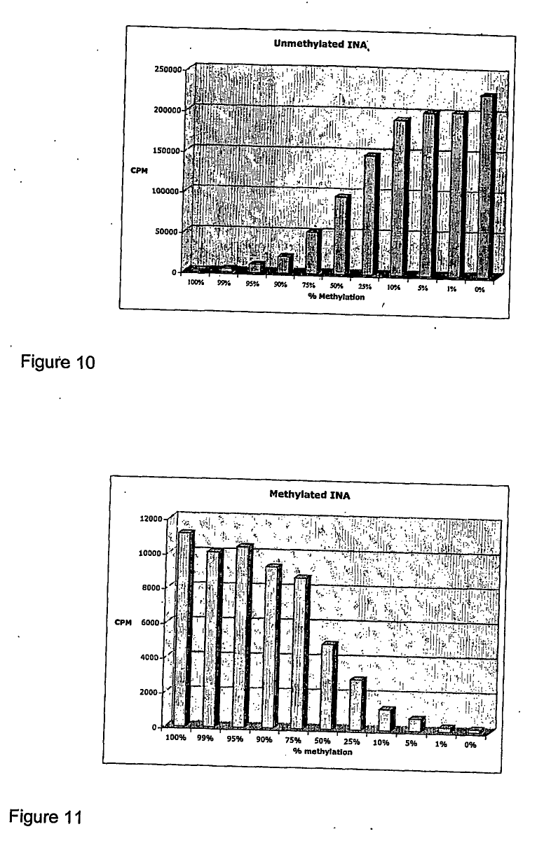

The results are shown in FIG. 10 and FIG. 11 where the specificity of an INA directed against unmethylated and methylated DNA, respectively, was demonstrated.

Specificity of INAs Versus PNAs Versus Oligonucleotides

Preparation of Capture Beads.

To determine the specificity of INA versus PNA versus oligonucleotide the following probes were synthesised.

INA Probe

(SEQ ID NO: 15)

5′ amine-YA TCY GGC YGC GCY AAC YTA Y
PNA Probe

(SEQ ID NO: 16)

5′ amine-ATC GCC GCG CAA CTA A
Oligo Probe

(SEQ ID NO: 17)

5′ amine-AAT CCC CGA AAT CGC CGC GCA ACT AA

The probes were synthesised to recognise a methylated sequence of the GSTP1 Gene (Accession number: M24485).
I. Ten μl of carboxylate modified Magnabind™ beads (Pierce cat#21353) were transferred to a clean 1.5 ml tube and 90 μl of PBS solution added to the magnetic beads.
II. The beads were mixed then magnetised and the supernatant discarded. The beads were washed ×2 in 100 μl of PBS per wash and finally resuspended in 90 μl of 50 mM MES buffer pH 4.5
III. One μl of 250 μM PNA was added to the sample and the tube vortexed and left at room temperature for 10-20 minutes.
IV. Ten μl of a freshly prepared 25 mg/ml EDC solution (Pierce/Sigma) is then added, the sample vortexed and incubated at either room temperature or 4° C. for up to 60 minutes.
V. The samples were then magnetised, the supernatant discarded.
VI. The beads were blocked by the addition of 100 μl either 0.25 M NaOH or 0.5 M Tris pH 8.0 for 10 minutes.
VII. The beads were then washed ×2 with PBS solution and finally resuspended in 100 μl PBS solution.
   Hybridisation of the Bead/Probe Complexes to a Synthetic GSTP1 Sequence.

A synthetic 110 bp oligo nucleotide was designed to represent a methylated region of the GSTP1 gene.

(SEQ ID NO: 18)

5′AGGGAATTTTTTTTCGCGATGTTTCGGCGCGTTAGTTCGTTGCGTATA
TTTCGTTGCGGTTTTTTTTTTGGTTTTTTCGGTTAGTTGCGCGGCGATTT
CGGGGATTTTAG-3′

The synthetic oligo was kinased at 1/10, 1/100 and 1/1,000 as follows

| | | |
|---|---|---|
| Oligo | 2 | μl |
| x10 buffer | 2 | μl |
| T4 Kinase | 2 | μl |
| Gamma P32 | 4 | μl |
| Water | to 20 | μl |

The reaction was heated at 37° C. for 60 minutes then the enzyme heat denatured at 95° C. for 5 minutes.

Hybridisation Reaction

I. 40 μl of ExpressHyb solution (Clontech) diluted 1:1 with distilled water was added to the 5 μl of magnetic bead coated probes in 1.5 ml centrifuge tubes.
II. 5 μl of kinased oligo was added and the solution mixed by vigorous pipetting to resuspend the particles.
III. The samples were then incubated at 55° C. for 30 minutes to allow binding of target sequences.
IV. The beads were magnetised and the supernatant removed.
V. The particles were then washed ×2 with ×2 SSC/0.1% SDS at 55° C. for 5 minute per wash.
VI. The beads were washed a further ×1 with ×1 SSC/0.1% SDS at 55° C. for 5 minute.
VII. The supernatant removed and finally the beads were resuspended in 5 ml of InstaGel scintillant and the radioactivity determined by scintillation counting using the Cerenkov protocol

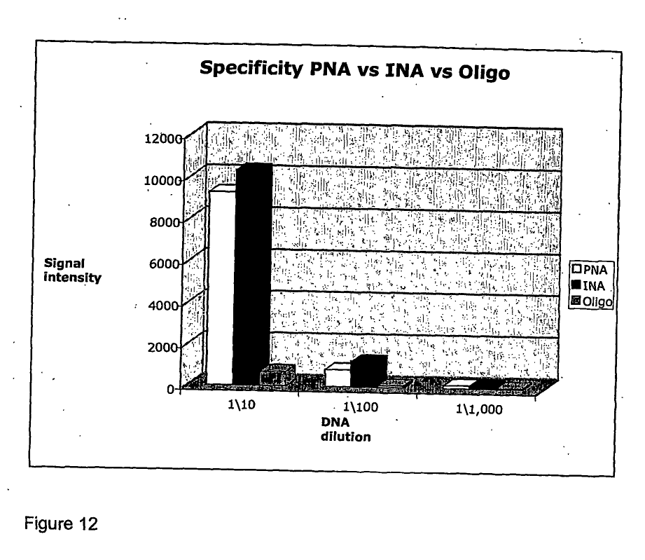

FIG. 12 shows the signals generated on hybridization of the PNA, INA and oligo samples with a synthetic 110 bp oligo designed to a methylated region of the GSTP1 gene. The oligo was diluted as described then labelled and hybridised to the samples. As can be seen the INA gave signal intensities similar if not higher than the PNA probe.

FIG. 12 shows the results generated when a PNA, INA and oligonucleotide were designed to detect an identical genomic locus. The PNA, INA and oligonucleotide ligands were hybridised with a serially diluted synthetic bisulphite converted sequence. After hybridization the samples were washed to remove unbound molecules then the remaining specific bound molecules quantified. As can be see the INA gave higher specificity than the PNA and over a 15 fold increase in detection signal intensity compared to that of the conventional oligonucleotide.

Comparison of INA Ligands Versus Oligonucleotides Using an Array Type Hybridisation on a Solid Support

To test the hybridisation of INA ligands versus oligonucleotides the following INA probes were synthesised to various gene loci whose symbols are given below M=methylated sequence detection, U=unmethylated sequence detection.

| | | |
|---|---|---|
| Probe | Sequence | SEQ ID No |

| | | |
|---|---|---|
| ABCB1-M | 2GTTTATTAAGACGTTTTATATTTTA | 19 |
| ABCB1-U | 2GTTTATTAAGATGTTTTATATTTTA | 20 |
| ABCG2-M | 2TTTTTGGATGTTCGGGTTTTTTTAG | 21 |
| ABCG2-U | 2TTTTTGGATGTTTGGGTTTTTTTAG | 22 |
| BRCA-M | 2TTGGGTTTTTGCGTTTAGGAGGTTT | 23 |
| BRCA-U | 2TTGGGTTTTTGTGTTTAGGAGGTTT | 24 |
| CD38-M | 2GTAATTAGTTACGGAATTTTGAGGT | 25 |
| CD38-U | 2GTAATTAGTTATGGAATTTTGAGGT | 26 |
| CFTR-M | 2GAAAAGGTTAGCGTTGTTTTTAAAT | 27 |
| CFTR-U | 2GATAAGGTTAGTGTTGTTTTTAAAT | 26 |
| EZH2-M | 2TTAGTTTGTTGCGGATTAAAATATA | 29 |
| EZH2-U | 2TTAGTTTGTTGTGGATTAAAATATA | 30. |
| MAGEA2-M | 2TTTATTTTTGTCGTGAATTTAGGGA | 31 |
| MAGEA2-U | 2TTTATTTTTGTTGTGAATTTAGGGA | 32 |
| PRKCDBP-M | 2GAAGGTTAATTTCGTTTGTTTGAGT | 33 |
| PRKCDBP-U | 2GAAGGTTAATTTTGTTTGTTTGAGT | 34 |
| PTGS2-M | 2AAAAGATATTTGGCGGAAATTTGTG | 35 |
| PTGS2-U | 2AAAAGATATTTGGTGGAAATTTGTG | 36 |
| RASSF1-U | 2TTATTGAGTTGTGGGAGTTGGTATT | 37 |
| RASSF1-M | 2TTATTGAGTTGCGCGAGTTGGTATT | 38 |

In order to compare the INA ligands with oligos, the same oligonucleotide sequences were synthesised.

PCR products were generated from each selected genomic region using a 10× multiplex reaction under standard conditions using the Qiagen Multiplex PCR kit (Qiagen P/N 206143)

Coupling the INA Ligands and Oligonucleotides to the Solid Support.

I. An 8 cm×12 cm section of Biodyne C transfer membrane (Pall P/70155A) was cut and rinsed briefly with 0.1 N HCl.
II. The membrane was soaked for 15 minutes in freshly prepared EDC solution (Sigma) in water.
III. The membrane was rinsed in water and placed into a 96 well dot blot apparatus.
IV. 500 ng of INA and oligo were diluted in 20 μl of PBS and pipetted into the appropriate wells.
V. The membrane was left for 10 minutes at room temperature and then a vacuum applied and the wells dried.
VI. The wells were then rinsed ×2 with 200 μl PBS/0.1% Tween 20, applying the vacuum between washes.
VII. The membrane was removed from the blotting apparatus and the remaining active sites on the membrane quenched with 0.1N NaOH for 10 minutes at room temperature.
VIII. The membrane was rinsed with distilled water and finally air dried for 30 minutes prior to-use.
   Preparation of the P³² Labelled Multiplex Probe

Radioactive probes were prepared using the Prime-a-gene Labelling system (Promega Cat#U 1100)

| | |
|---|---|
| PCR products | 2 μl |
| PCR grade water | 21 μl |

The sample was heated 95° C. for 5 minutes then snap chilled on ice.

| | |
|---|---|
| dNTP mix | 6 μl |
| Primer mix | 15 μl |
| P³²dATP | 5 μl |
| Klenow | 1 μl |

The probe was left at room temperature for 1 hour then purified using the wizard DNA clean-up system according to the manufacturers instructions.

Prehybridisation/Hybridisation of Coated Membrane

I. The membrane was prehybridised in 10 ml of ExpressHyb solution (Clontech) containing 100 μg/ml sheared salmon testis DNA (Sigma) in roller bottles at 55° C. rotating at 7 rpm per minute for 1 hour.
II. The probe was boiled for 5 minutes, snap chilled on ice for 5 minutes then added to the membrane.
III. Hybridisation was carried out overnight at 55° C. in bottles rotating at 7 rpm per minute.
   Washing the Membrane
I. The membrane was then washed ×2 with ×2 SSC/0.1% SDS at 55° C. for 20 minutes per wash.
II. The membrane was washed a further ×1 with ×1 SSC/0.1% SDS at 50° C. for 20 minutes.
III. Finally the membrane was washed ×1 with ×0.1 SSC/0.1% SDS at 55° C. for 20 minutes.
IV. The membrane was wrapped in glad wrap and exposed to a Molecular Dynamics phosphorimager.

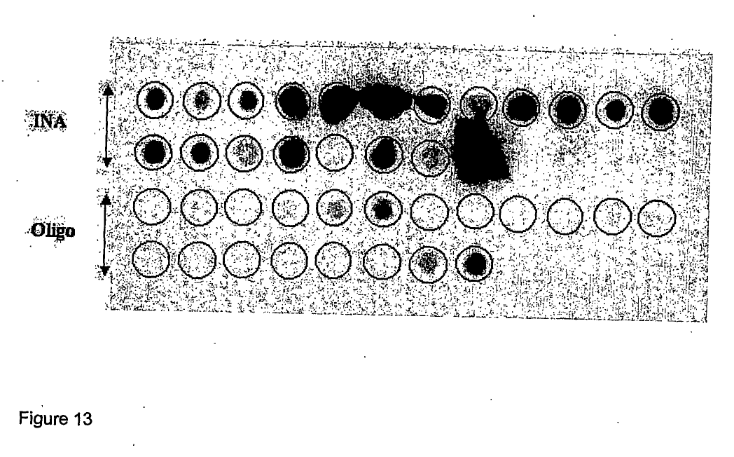

Hybridisation results using INAs versus conventional oligonucleotides are set out in FIG. 13. Top two rows signals generated using INAs. Bottom two rows signals generated using conventional oligonucleotides. From FIG. 13 the superior quality of the hybridisation signals generated using INAs can be clearly seen.

Advantages of INA Over PNA

INA ligands can be synthesised on standard oligonucleotide platform whereas PNA ligands have to be synthesised on specialised peptide synthesis machines.

PNA ligands cannot be used as primers in standard molecular techniques such as PCR, reverse transcription, real time PCR, isothermal amplification reactions, extension reactions. In contrast, INA ligands can be used in all of the above making them much more useful tools for molecular biology.

INA ligands can be made so they are exonuclease resistant.

INA ligands can be designed to selectively bind to DNA whereas PNA ligands bind to both DNA and RNA.

INA ligands also exhibit endogenous fluorescence making them useful molecules in application such as real time PCR, whereas PNA ligands do not.

INA ligands also have decreased self-affinity when compared to PNA ligands.

The present inventors have found that PNA ligands are also rather “sticky” in that they seem to stick non-specifically to surfaces. This is especially evident when two INA ligands are used in the same system. INA ligands do not seem to suffer from this problem.

Summary

The methods of the present invention can be applied for the detection of any DNA using one ligand (preferably an oligonucleotide or INA) bound to a solid support and one coupled to a microsphere. Natural oligonucleotides or INAs may be used, but INAs were preferred because of their specificity, stability and rate of hybridisation.

In one particular adaptation, the methods of the invention can be used to distinguish the presence of methylated cytosines in DNA that has been treated with sodium bisulfite. The specificity of hybridisation can be used to discriminate against molecules that have not reacted completely with bisulfite (one or more cytosines not converted to uracil) as well as distinguishing between methylated cytosines at CpG sites (which remain as cytosines) and unmethylated CpG sites where the cytosine is converted to uracil.

In another adaptation the methods of the invention can be used to discriminate against DNA whose cytosines have not reacted completely with bisulfite reagent to convert them to uracils.

As treatment with bisulfite changes the sequence of the DNA by converting all cytosines (but not 5-methyl cytosines) to uracils, specific INAs can be made which recognise a region having 5 methyl cytosines but which will not recognise the same sequence which happens to have no 5-methyl cytosines.

The methods of the invention can also be applied to the discrimination of different alleles of a gene where the sequence of one or both of the oligonucleotides or INAs will match perfectly with one allele but mismatch with the other.

The method of the invention has numerous applications as previously described including particular use in devising multiple array chips for rapid detection of the methylation status of bulk DNA samples. Detectable particles can also be used to scale up and automate the detection and screening process.

It will be appreciated that the methods are applicable for many other states and conditions where different methylation states have been found to play a role in disease or altered state of cells. Examples of just some genes affected by CpG methylation are shown in Table 3. The present invention is clearly applicable for the detection or measurement of such methylation states and many others.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

TABLE 3
Examples of genes affected by CpG or CpNpG methylation

| Gene | Location | Cancer | Aging | Comments |
|---|---|---|---|---|
| APC | 5q21 | Colon, gastric, | No | |
| | | oesophageal | | |
| BRCA-1 | 17q21 | Breast, ovarian | No | |
| Calcitonin | 11p15 | Colon, lung, | No | One of the first to be |
| | | haematological | | found methylated in |
| | | | | cancer |
| E-cadherin | 16q22.1 | Breast, gastric, | No | |
| | | thyroid, SCC, | | |
| | | leukemia, liver | | |
| Estrogen | 6q25.1 | Colon, liver, heart, breast, | Yes | Good correlation |
| Receptor | | lung | | between methylation and |
| | | | | loss of expression |
| H19 | 11p15.5 | Wilms tumour | No | Imprinted gene |
| HIC1 | 19p13.3 | Prostate, breast, | Yes | Candidate tumour |
| | | brain, lung | | suppressor |
| IGF2 | 11p15.5 | Colon, AML | Yes | Has large CpG island |
| MDGI | 1p33-35 | Breast | No | |
| MGMT | 10q26 | Brain, colon, lung, | No | |
| | | breast | | |
| MYOD1 | 11p15.4 | Colon, breast, | Yes | |
| | | bladder, lung | | |
| N33 | 8p22 | Colon, prostate, | Yes | Oligo-saccharyl- |
| | | brain | | transferase |
| p15 | 9q21 | Leukemia, lung, | No | |
| | | colon | | |
| p16 | 9q21 | Lung, colon, | No | Methylation occurs as |
| | | lymphoma, bladder, | | frequently as deletions or |
| | | and more | | other mutations |
| TIMP3 | 22q12.1 | Brain, kidney | No | |
| WT1 | 11p13 | Breast, colon, Wilms | No | |
| | | tumour | | |

1-32. (canceled)

33. A method for detecting the presence of a target nucleic acid in a sample comprising:
treating a sample containing nucleic acid with an agent that modifies unmethylated cytosine;
providing to the treated sample a detector ligand in the form of an intercalating nucleic acid (INA) capable of binding to a target region of nucleic acid, and allowing sufficient time for the detector ligand to bind to the target nucleic acid; and
detecting binding of the detector ligand to nucleic acid molecule in the sample to indicate the presence of the target nucleic acid.

34. The method according to claim 33 wherein the nucleic acid is obtained from a genome of an eukaryote, a prokaryote, virus, mitochondrial nucleic acid, nucleic acid found in other cellular organelles, extracellular nucleic acid, DNA and RNA forms and natural or artificial derivatives of DNA and RNA.

35. The method according to claim 34 wherein the natural or artificial derivatives of DNA and RNA are selected from the group consisting of INA, ANA, MNA, PNA, LNA, HNA, CNA, and chimeric.

36. The method combinations thereof according to claim 34 wherein the nucleic acid is genomic DNA.

37. The method according to claim 33 wherein the agent is selected from bisulfite, acetate or citrate.

38. The method according to claim 37 wherein the agent is sodium bisulfite, a reagent, which in the presence of water, modifies cytosine into uracil.

39. The method according to claim 33 wherein the INA is phosphoramidite of (S)-1-O-(4,4′-dimethoxytriphenylmethyl)-3-O-(1-pyrenylmethyl)-glycerol.

40. The method according to claim 33 wherein the target region includes at least one 5′-methyl cytosine in the untreated nucleic acid.

41. The method according to claim 33 wherein the detector ligand is directed to a CpG- or CpNpG-containing region of DNA, where N designates any one of the four possible bases A, T, C, or G.

42. The method according to claim 41 wherein the CpG-, or CpNpG-containing region of DNA is in a regulatory region of a gene or an enhancer of any regulatory element or region including promoter, enhancer, oncogene, retro-element, mobile or mobilisable sequence or other regulatory element which activity is altered by environmental factors including chemicals, toxins, drugs, radiation, synthetic or natural compounds and microorganisms or other infectious agents such as viruses, bacteria, fungi and prions.

43. The method according to claim 33 wherein prior to treating the sample, the nucleic acid is undergoes an enrichment or selection step.

44. The method according to claim 42 wherein the enrichment or selection step is selected from the group consisting of physical methods including sonication and shearing, enzymatic digestion, enzymatic treatment, restriction digestion, nuclease treatment, Dnase treatment, concentration, antibody capture, chemical methods including acidic or base digestion and combinations thereof.

45. The method according to claim 43 wherein the enrichment or selection step is treatment with an antibody directed to 5′-methyl cytosine so as to obtain a methylated nucleic acid sample.

46. The method according to claim 33 wherein the method detects methylation of a target nucleic acid by providing to the treated sample a detector ligand in the form of an intercalating nucleic acid (INA) capable of distinguishing between methylated and unmethylated cytosine of nucleic acid, such that detection of binding of the detector ligand to the nucleic acid in the sample is indicative of the extent of methylation of the target nucleic acid.

47. The method according to claim 33 wherein a capture ligand capable of recognising a first part of a target nucleic acid sequence is bound to a solid support such that the treated nucleic acid binds to the support via the first capture ligand, the bound nucleic acid is then exposed to a detector ligand capable of recognising a second part of the target nucleic acid sequence and allowing sufficient time for the detector ligand to bind to a target nucleic acid bound to a support wherein binding of the detector ligand to nucleic acid bound to the support is measured to determine the presence of the target nucleic acid in the sample, wherein at least one of the capture ligand or the detector ligand is an INA ligand.

48. The method according to claim 47 wherein the ligands are selected from the group consisting of INA probe, peptide nucleic acid (PNA) probe, LNA probe, HNA probe, ANA probe, MNA probe, oligonucleotide, modified oligonucleotide, single stranded DNA, RNA, aptamer, antibody, protein, peptide, a combination thereof, and chimeric versions thereof.

49. The method according to claim 48 wherein the capture ligand is selected from the group consisting of INA probe, PNA probe, and oligonucleotide probe.

50. The method according to claim 47 wherein both the capture ligand and the detector ligand are an INA ligand.

51. The method according to claim 47 wherein the detector ligand is an INA ligand capable of distinguishing between methylated and unmethylated cytosine of DNA and the degree or amount of binding of the detector ligand is indicative of the extent of methylation of the target nucleic acid.

52. The method according to claim 47 wherein the support is selected from the group consisting of plastic materials, fluorescent beads, magnetic beads, shaped particles, plates, microtiter plates, synthetic or natural membranes, latex beads, polystyrene, column supports, glass beads or slides, nanotubes, arrays, fibres, organic, and inorganic supports.

53. The method according to claim 52 wherein the support is a magnetic bead, a fluorescent bead, a shaped particle, bead array, or a microtiter plate with one or more wells.

54. The method according to claim 47 wherein a plurality of capture ligands are arrayed on the solid support.

55. The method according to claim 33 wherein the INA detector ligand has a detectable label attached thereto.

56. The method according to claim 55 wherein detectable label is selected from the group consisting of chemiluminescence, fluorescence, radioactivity, enzyme, hapten, and dendrimer.

57. The method according to claim 33 wherein the nucleic acid bound to the INA detector ligand is further processed or treated.

58. The method according to claim 57 wherein the nucleic acid is amplified using polymerase chain reaction using primers directed to regions of nucleic acid.

59. The method according to claim 58 wherein the primers are INA ligands.

60. A kit for use in analysing nucleic acid which has been treated with an agent that modifies unmethylated cytosine according to the method of claim 33 comprising at least one INA ligand capable of distinguishing between methylated and unmethylated cytosine of DNA.

61. The kit according to claim 60 wherein one or more INA ligands are immobilized to a solid support.

62. The kit according to claim 61 wherein the solid support is selected from the group consisting of plastic materials, fluorescent beads, magnetic beads, shaped particles, plates, microtiter plates, synthetic or natural membranes, latex beads, polystyrene, column supports, glass beads or slides, nanotubes, arrays, fibres, organic, and inorganic supports.

63. The kit according to claim 60 further comprising primers for amplifying treated DNA.

64. The kit according to claim 63 wherein the primers are INA primers.
